(19) United States
(12) Patent Application Publication
(10) Pub. No.: US 2025/0230161 A1
(43) Pub. Date: Jul. 17, 2025

(54) POLYCYCLIC CAP-DEPENDENT ENDONUCLEASE INHIBITORS FOR TREATING OR PREVENTING INFLUENZA

(71) Applicants: Merck Sharp & Dohme LLC, Rahway, NJ (US); MSD R&D (China) CO., LTD., Beijing (CN); COCRYSTAL PHARMA, INC., Bothell, WA (US)

(72) Inventors: Valerie W. Shurtleff, Philadelphia, PA (US); Brendan M. Crowley, Collegeville, PA (US); James I. Fells, Lansdale, PA (US); Ronald M. Kim, Summit, NJ (US); Jennie Liao, Philadelphia, PA (US); John A. McCauley, Maple Glen, PA (US); John D. Schreier, Harleysville, PA (US); Hua-Poo Su, Phoenixville, PA (US); Yonglian Zhang, East Brunswick, NJ (US); Lianyun Zhao, Shanghai (CN)

(21) Appl. No.: 18/682,331

(22) PCT Filed: Aug. 11, 2022
(86) PCT No.: PCT/US2022/040127

§ 371 Date: Feb. 8, 2024

Related U.S. Application Data
(60) Provisional application No. 63/231,871, filed on Aug. 11, 2021.

(51) Int. Cl. C07D 471/18; A61K 31/53; A61P 31/16

(52) U.S. Cl. CPC C07D 471/18; A61K 31/53; A61P 31/16

(57) ABSTRACT

The present invention provides a compound of Formula I

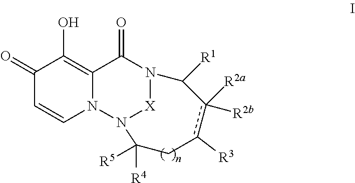

and pharmaceutical compositions comprising one or more said compounds, and methods for using said compounds for treating or preventing influenza. The compounds are cap-dependent endonuclease inhibitors.

### BACKGROUND OF THE INVENTION

Influenza viruses, members of the Orthomyxoviridae family, are categorized by type: Influenza A, B, C or D. Seasonal epidemic disease caused by Influenza A and Influenza B, which co-circulate throughout the world, is the biggest concern for human public health. Influenza A viruses are characterized by the combination of surface proteins, hemagglutinin (HA, H) and neuraminidase (NA, N), present on the virion. Both H1N1 and H3N2 viruses are capable of infecting and causing disease in humans. Influenza B viruses fall into one of two lineages, Victoria-like or Yamagata-like, both of which cause human disease.

Influenza A and B virus particles consist of a cell-derived lipid membrane lined with the viral M1 matrix protein. This envelope encompasses 8 segments of negative strand RNA genome, each encoding one or more viral proteins. Surface-exposed hemagglutinin, M2 and neuraminidase proteins mediate host cell entry, uncoating and release of nascent virus particles from infected cells, respectively. The segmented genome is packaged as a ribonucleoprotein complex made up of nucleoprotein-coated RNA associated with the heterotrimeric polymerase. The polymerase, made up of PA, PB1 and PB2 subunits, is critical for both viral genome replication and mRNA transcription. The PB1 subunit harbors the polymerase active site, while the PB2 and PA subunits, in addition to their role in genome replication, work together to capture (PB2) and remove the cap (PA) from host cell pre-mRNAs, facilitating transcription of viral mRNA.

Seasonal influenza is a respiratory disease characterized by sudden onset fever, cough, sore throat, headache, myalgia, and malaise. Symptoms range from mild to severe and may lead to death of the infected person. Worldwide, 3-5 million people each year suffer from severe influenza disease and approximately 0.5 million die. Those who are immune-compromised, including the very young and those over the age of 65, are at highest risk for influenza-related morbidity and mortality.

Vaccines for prevention of influenza disease are available; however, the effectiveness of such vaccines varies from year to year with an estimated pooled effectiveness of 59% for healthy adults (Osterholm et al, CIDRAP report (2012)). Influenza virus strains capable of escaping host immunity are selectively transmitted; thus, to provide protection against currently circulating virus, seasonal influenza vaccines must be reformulated and readministered annually. Vaccines that provide durable, multi-season or broad spectrum protection, are not currently available.

Several small molecules targeting influenza virus have been approved for therapeutic and/or limited prophylactic use in one or more countries, including M2 ion channel inhibitors, NA inhibitors, a nucleoside analog and a recently approved inhibitor targeting the endonuclease activity of the PA protein. As therapy, small molecule inhibitors of influenza must be administered within 48 hours of symptom onset to be effective, and shorten the duration of virus shedding and respiratory symptoms. Currently circulating influenza virus strains are resistant to approved M2 inhibitors, such that use of M2 inhibitors is no longer recommended. The purine analog favipiravir is approved for use only in Japan, and safety concerns restrict its use. In the past, increasing levels of circulating drug-resistant virus variants had limited the effectiveness of several neuraminidase inhibitors; however, since the 2009 H1N1 pandemic, the level of drug resistance has been low. Resistance to the cap-dependent endonuclease inhibitor baloxavir marboxil was high in late stage clinical trials wherein virus harboring a mutation at amino acid 38 of the PA protein was isolated from 9.7% of adult and 23.4% of pediatric trial participants (Hayden et al, N Engl J Med., 379(10):913-923 (2018); Hirotsu et al, Clin Infect Dis., ciz908 (2019)). Whether these mutant viruses, which significantly reduce the in vitro antiviral potency, can efficiently transmit from human to human is not known (Omoto et al, Sci Rep., 8(1):9633 (2018); Noshi et al, Antiviral Res., 160:109-117 (2018)).

Currently, there are limited options for the treatment and prevention of influenza disease and significant concerns about drug resistance with approved therapies. Furthermore, the inability of seasonal vaccines to provide consistent, robust, durable, broad spectrum protection against influenza disease, combined with the threat of emergence of novel zoonotic influenza viruses with pandemic potential, necessitate continued development of both prophylactic and therapeutic agents targeting the influenza virus.

### SUMMARY OF THE INVENTION

The present invention relates to compounds of Formula I:

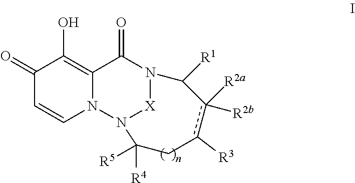

and pharmaceutically acceptable salts thereof. The compounds of Formula I are cap-dependent endonuclease inhibitors, and as such may be useful in the treatment, inhibition or amelioration of one or more disease states that could benefit from inhibition of a virus having a cap-dependent endonuclease, including influenza. The compounds of this invention could further be used in combination with other therapeutically effective agents, including but not limited to, other drugs useful for the treatment of influenza. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

The present invention relates to compounds of Formula I.

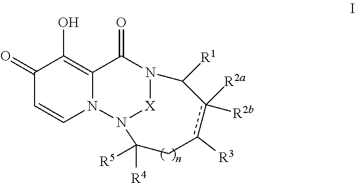

wherein --- is an optional double bond;
X is CHR⁶;
R¹ is selected from the group consisting of hydrogen, halo, hydroxy, C₁₋₆ alkyl and C₃₋₆ cycloalkyl;
R^{2a} is selected from the group consisting of hydrogen, halo, hydroxy and C₁₋₆ alkyl;
R^{2b} is hydrogen or halo, but when --- is a double bond, R^{2b} is absent;
R³ is selected from the group consisting of hydrogen, halo, hydroxy and C₁₋₆ alkyl;
R⁴ is selected from the group consisting of hydrogen and C₁₋₆ alkyl;
R⁵ is aryl, which may be monocyclic or bicyclic, which optionally substituted with one to three substituents independently selected from the group consisting of halo, R^{x} and OR^{x};
R⁶ is hydrogen or C₁₋₃ alkyl;
R^{x} is selected from the group consisting of hydrogen and C₁₋₆ alkyl, wherein said alkyl is optionally substituted with one to three halo;
n is one or two;
or a pharmaceutically acceptable salt thereof.

In an embodiment of the invention, --- is a double bond. In another embodiment of the invention, --- is not a double bond.

In an embodiment of the invention, R¹ is hydrogen, fluoro, methyl or cyclopropyl. In a class of the embodiment, R¹ is hydrogen. In another class of the embodiment, R¹ is fluoro. In another class of the embodiment, R¹ is methyl. In another class of the embodiment, R¹ is cyclopropyl.

In an embodiment of the invention, R^{2a} is hydrogen or fluoro. In a class of the embodiment, R^{2a} is hydrogen. In another class of the embodiment, R^{2a} is fluoro.

In an embodiment of the invention, R^{2b} is hydrogen or fluoro. In a class of the embodiment, R^{2b} is hydrogen. In another class of the embodiment, R^{2b} is fluoro.

In an embodiment of the invention, R³ is hydrogen, methyl or hydroxy. In a class of the embodiment, R³ is hydrogen. In another class of the embodiment, R³ is methyl. In another class of the embodiment, R³ is hydroxy.

In an embodiment of the invention, R⁴ is hydrogen or methyl. In a class of the embodiment, R⁴ is hydrogen. In another class of the embodiment, R⁴ is methyl.

In an embodiment of the invention, R⁵ is phenyl or naphthalenyl, wherein said phenyl and naphthalenyl are optionally substituted with halo. In a class of the embodiment, R⁵ is phenyl, which is optionally substituted with halo. In another class of the embodiment, R⁵ is naphthalenyl, which is optionally substituted with halo.

In an embodiment of the invention, R⁶ is hydrogen or methyl. In a class of the embodiment, R⁶ is hydrogen. In another class of the embodiment, R⁶ is methyl.

In an embodiment of the invention, n is one. In another embodiment of the invention, n is two.

Reference to the preferred classes and subclasses set forth above is meant to include all combinations of particular and preferred groups unless stated otherwise.

Specific embodiments of the present invention include, but are not limited to compounds 1 to 23 identified herein as Examples 1 to 13, or pharmaceutically acceptable salts thereof.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

The invention also includes compositions for inhibiting cap-dependent endonuclease in a virus, treating a disease caused by a virus having a cap-dependent endonuclease, treating influenza and preventing influenza, in a mammal, comprising a compound of the invention in a pharmaceutically acceptable carrier. These compositions may optionally include other antiviral agents. The compositions can be added to blood, blood products, or mammalian organs in order to effect the desired inhibitions.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Also included are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups that may be quatemized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

If the compounds of Formula I simultaneously contain acidic and basic groups in the molecule the invention also includes, in addition to the salt forms mentioned, inner salts or betaines (zwitterions).

The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both each individual enantiomer and mixtures thereof, are embraced within the Formula. When a particular configuration is depicted, that entantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

Unless a specific enantiomer or diastereomer is indicated, the invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the transform as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (1_{H}) and deuterium (2_{H}). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings: A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “preventing,” or “prophylaxis,” as used herein, refers to reducing the likelihood of contracting disease or disorder described herein, or reducing the severity of a disease or disorder described herein.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “haloalkyl,” as used herein refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “halo,” as used herein, means —F, —Cl, —Br or —I.

The term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on. Bicyclic cycloalkyl ring systems include fused ring systems, where two rings share two atoms, and spiro ring systems, where two rings share one atom.

The term “aryl”, as used herein, represents a stable monocyclic or bicyclic ring system of up to 10 atoms in each ring, wherein at least one ring is aromatic, and all of the ring atoms are carbon. Bicyclic ring systems include fused ring systems, where two rings share two atoms, and spiro ring systems, where two rings share one atom.

“Celite®” (Fluka) diatomite is diatomaceous earth, and can be referred to as “celite”.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., R^{x}) occurs more than one time in any constituent or in Formula I, its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

The invention also relates to medicaments containing at least one compound of the Formula I and/or of a pharmaceutically acceptable salt of the compound of the Formula I and/or an optionally stereoisomeric form of the compound of the Formula I or a pharmaceutically acceptable salt of the stereoisomeric form of the compound of Formula I, together with a pharmaceutically suitable and pharmaceutically acceptable vehicle, additive and/or other active substances and auxiliaries.

The term “patient” used herein is taken to mean mammals such as primates, humans, sheep, horses, cattle, pigs, dogs, cats, rats, and mice.

The term “influenza” includes seasonal influenza, pandemic influenza, avian influenza, swine influenza and influenza disease in humans or animals. Seasonal influenza is caused by Influenza A and/or Influenza B viruses.

The medicaments according to the invention can be administered by oral, inhalative, rectal or transdermal administration or by subcutaneous, intraarticular, intraperitoneal or intravenous injection. Oral administration is preferred. Coating of stents with compounds of the Formula (I) and other surfaces which come into contact with blood in the body is possible.

The invention also relates to a process for the production of a medicament, which comprises bringing at least one compound of the Formula (I) into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the cap-dependent endonuclease inhibitors of the instant invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the cap-dependent endonuclease inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the cap-dependent endonuclease inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/mL, e.g. 0.1 mg/mL, 0.3 mg/mL, and 0.6 mg/mL, and administered in amounts per day of between 0.01 mL/kg patient weight and 10.0 mL/kg patient weight, e.g. 0.1 mL/kg, 0.2 mL/kg, 0.5 mL/kg. In one example, an 80 kg patient, receiving 8 mL twice a day of an intravenous formulation having a concentration of active ingredient of 0.5 mg/mL, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

Compounds of Formula I can be administered both as a monotherapy and in combination with other therapeutic agents, including other antivirals or treatments of influenza.

The cap-dependent endonuclease inhibitors can also be co-administered with suitable antivirals, including, but not limited to, M2 ion channel inhibitors, neuraminidase inhibitors, nucleoside analogs and inhibitors targeting the endonuclease activity of the PA protein.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to M2 ion channel inhibitors, neuraminidase inhibitors, nucleoside analogs and inhibitors targeting the endonuclease activity of the PA protein may be used in any combination with the compound of the invention in a single dosage formulation (a fixed dose drug combination), or may be administered to the patient in one or more separate dosage formulations which allows for concurrent or sequential administration of the active agents (co-administration of the separate active agents).

Typical doses of the cap-dependent endonuclease inhibitors of the invention in combination with other suitable M2 ion channel inhibitors, neuraminidase inhibitors, nucleoside analogs and inhibitors targeting the endonuclease activity of the PA protein may be the same as those doses of the cap-dependent endonuclease inhibitors administered without coadministration of additional M2 ion channel inhibitors, neuraminidase inhibitors, nucleoside analogs and inhibitors targeting the endonuclease activity of the PA protein, or may be substantially less that those doses of thrombin inhibitors administered without coadministration of M2 ion channel inhibitors, neuraminidase inhibitors, nucleoside analogs and inhibitors targeting the endonuclease activity of the PA protein, depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e., prevent, inhibit or ameliorate) the viral condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

### GENERAL METHODS

Several methods for preparing the compounds of this invention are illustrated in the following Schemes and Examples. Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are carried out by employing reactions as shown in the schemes and examples herein, in addition to other standard manipulations such as ester hydrolysis, cleavage of protecting groups, etc., as may be known in the literature or exemplified in the experimental procedures. Starting materials are made according to procedures known in the art or as illustrated herein.

The compounds of the present invention can be prepared in a variety of fashions. In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. Because the schemes are an illustration, the invention should not be construed as being limited by the chemical reactions and conditions expressed. The preparation of the various starting materials used herein is well within the skill of a person versed in the art. The following examples are provided so that the invention might be more fully understood. These examples are illustrative only and should not be construed as limiting the invention in any way. Absolute stereochemistry of separate stereoisomers in the examples and intermediates are not determined unless stated otherwise in an example or explicitly in the nomenclature.

In the methods for preparing compounds of the present invention set forth in the foregoing schemes, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection may be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively, the interfering group may be introduced into the molecule subsequent to the reaction Step of concern.

One skilled in the art of organic synthesis will recognize that the synthesis of compounds with multiple reactive functional groups, such as —OH and NH₂, may require protection of certain functional groups (i.e., derivatization for the purpose of chemical compatibility with a reaction condition). Suitable protecting groups for the various functional groups of these compounds and methods for their installation and removal are well-known in the art of organic chemistry. A summary of many of these methods may be found in Greene & Wuts, Protecting Groups in Organic Synthesis, John Wiley & Sons, 3^{rd} edition (1999).

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

The starting materials used, and the intermediates prepared using the methods set forth in Schemes 1 to 4 may be isolated and purified if desired using conventional techniques, including but not limited to filtration, distillation, crystallization, chromatography and alike. Such materials may be characterized using conventional means, including physical constants and spectral data.

### GENERAL SCHEMES

### Methods For Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Schemes and Examples below. Alternative synthetic pathways and analogous structures will be apparent to those skilled in the art of organic synthesis.

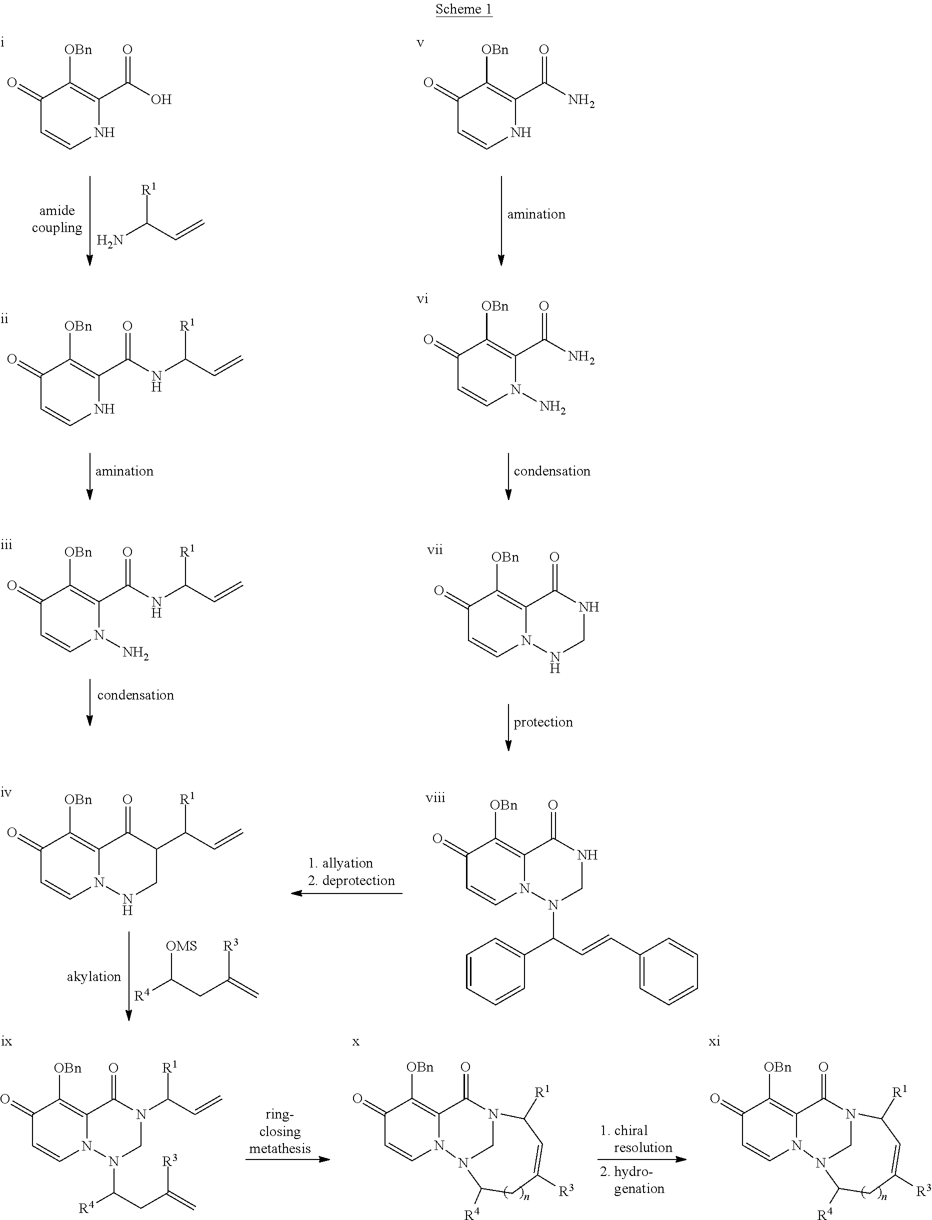

Pyridinone compound ii may be formed by amide coupling of compound i with an appropriate allylamine. Amination of the pyridinone affords a compound of formula iii, which may be condensed with formaldehyde to provide a bicyclic compound of formula iv. Alternatively, compound v may be aminated to provide the primary amide vi, which upon condensation with formaldehyde provides compound vii. Protection affords compound viii, which may be allylated and deprotected to afford a compound of formula iv. A compound of formula iv may be alkylated using an allyl mesylate to provide a compound of formula ix. Ring-closing metathesis affords a tricyclic compound of formula x. Chiral resolution and hydrogenation delivers a compound of formula xi, which corresponds to a compound of formula (I).

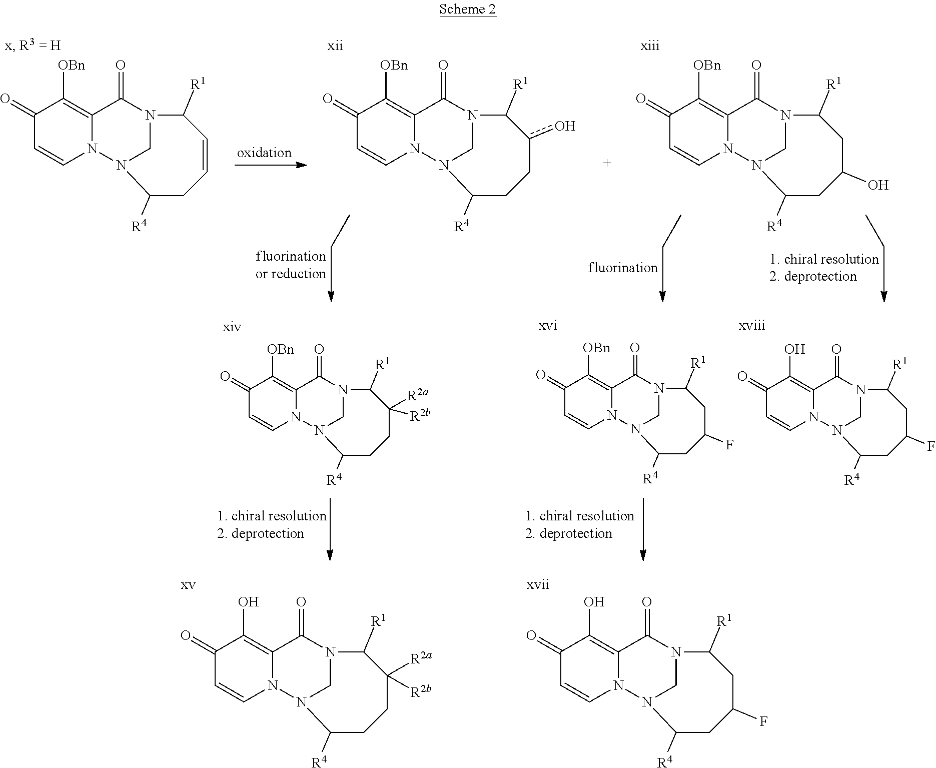

Compounds of formula xii and xiii may be formed by oxidation of a compound of formula x with R³ and R¹═H. Fluorination or reduction afford compounds of formula xiv. Chiral resolution and deprotection produce compounds of formula xv. A compound of formula xvi may be prepared by fluorination of an alcohol of formula xiii. Subsequent chiral resolution and deprotection delivers a compound of formula xvii. Direct chiral resolution and deprotection of a compound of formula xiii delivers an alcohol of formula xvii.

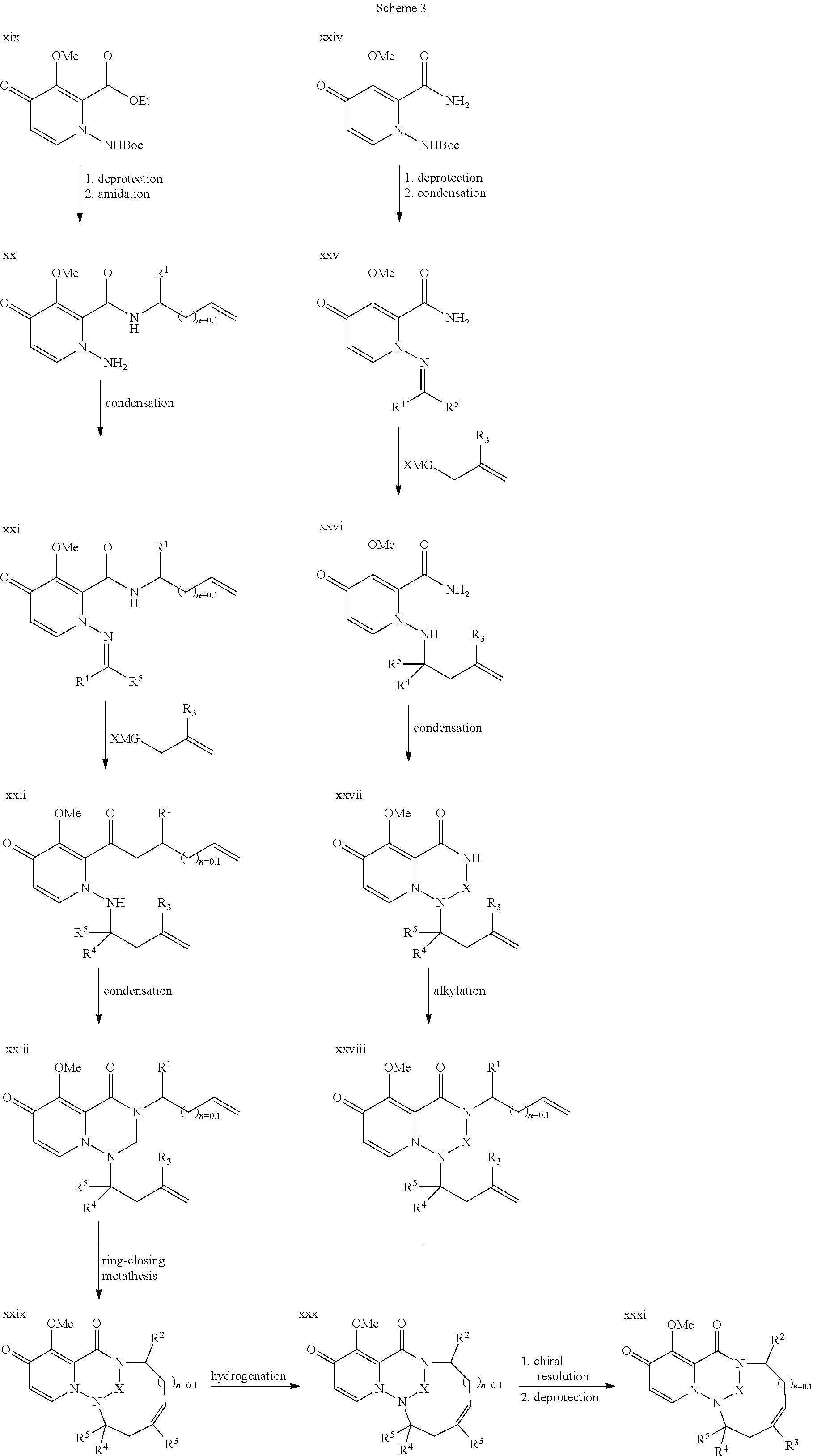

Sequential deprotection and amidation of ester xix provides a compound of formula xx. Such a compound may be condensed with a ketone or an aldehyde to provide a compound of formula xxi. Treatment with a Grignard reagent affords a compound of formula xxii. Condensation with formaldehyde yields a compound of formula xxiii. Ring-closing metathesis closes the tricyclic system leading to a compound of formula xxix. Alternatively, amide xxiv may be deprotected and condensed with a ketone or an aldehyde to provide a compound of formula xxv. Grignard addition affords a compound of formula xxvi, which may be condensed with formaldehyde or acetaldehyde to yield a compound of formula xxvii. Alkylation leads to a compound of formula xviii, which may be cyclized via ring-closing metathesis to afford a compound of formula xxix. Such a compound may be hydrogenated to form a saturated compound of formula xxx. Chiral resolution followed by deprotection provides a compound of formula xxxi.

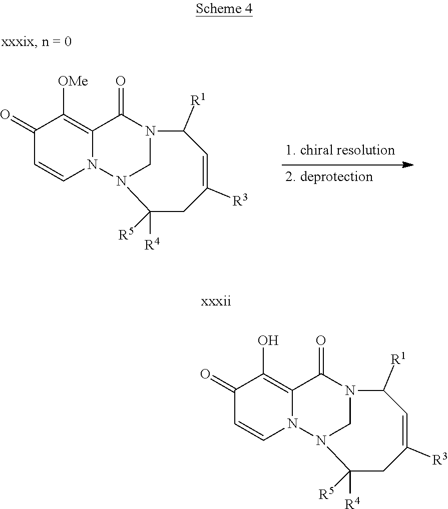

A compound of formula xxxix may be prepared following the method described in Scheme 3. Chiral resolution followed by deprotection provides a compound of formula xxxii, which corresponds to a compound of formula (I).

The following abbreviations may be used in the following experimentals:

| | |
|---|---|
| ° C. | Degrees Celsius |
| ACN | Acetonitrile |
| AcOH | Acetic acid |
| aq. | Aqueous |
| Atm | Atmospheres |
| BAST | 1 Bis(2-methoxtethyl)aminosulfur trifluoride |
| 9-BBN | 9-Borabicyclo [3.3.1]nonane |
| BHT | 3,5-Di-tert-4-butylhydroxytoluene |
| BSA | N,O-Bis(trimethylsilyl)acetamide |
| CDI | 1,1′-Carbonyldiimidazole |
| CD₃OD | Deuterated Methanol-d4 |
| CPME | Cyclopentyl methyl ether |
| DAST | Diethy laminosulfur trifluoride |
| DBU | Diazabicycloundecene |
| DCC | N,N′-Dicyclohexylcarbodiimide |
| DCE | 1,2-Dichloroethane |
| DCM | Dichloromethane |
| DDQ | 2,3-Dichloro-5,6-dicyano-p-benzoquinone |
| DEA | Diethylamine |
| DIAD | Diisopropyl azodicarboxylate |
| DIBAL | Diisobutylaluminium hydride |
| DIEA | N,N-Diisopropylethylamine |
| DIPA | Diisopropylamine |
| DIPEA | N,N-Diisopropylethylamine |
| DMA | N,N-Dimethylacetamide |
| DMAP | 4-Dimethylaminopyridine |
| DME | Dimethoxyethane |
| DMF | Dimethylformamide |
| DMP | Dess-Martin periodinane |
| DMPU | 1,3-Dimethyl-3,4,5,6-tetrahydro-2(1H)-pyrimidinone |
| DMSO | Dimethyl Sulfoxide |
| DMSO-d6 | Deuterated Dimethyl Sulfoxide |
| DPP | Diphenylphosphine |
| Dppf | Bis(diphenylphosphino)ferrocene |
| ESI | Electrospray Ionization |
| Et₃N | Triethylamine |
| Et₂O | Diethylether |
| EtOAc | Ethyl Acetate |
| EtOH | Ethanol |
| h | Hours |
| HATU | N-[(Dimethylamino)-1H-1,2,3-triazolo-[4,5- |
| | b]pyridin-1-ylmethylene]-N-methylmethanaminium |
| | hexafluorophosphate N-oxide |
| HPLC | High Performance Liquid Chromatography |
| IPA | Isopropyl alcohol |
| LAH | Lithium Aluminum Hydride |
| LED | Light-emitting diode |
| LiHMDS | Lithium bis(trimethylsilyl)amide |
| M | Molar |
| MeOD-d4 | Deuterated Methanol |
| MeOH | Methanol |
| MHz | Megahertz |
| Min | Minutes |
| Mn(TMHD)₃ | Tris(2,2,6,6-tetramethyl-3,5- |
| | heptanedionato)manganese(III) |
| mL | Milliliters |
| MP TMT resin | Macroporous polystyrene-bound trimercaptotriazine, |
| | a resin bound equivalent of 2,4,6-trimercaptotriazine |
| MS | Mass Spectroscopy |
| MTBE | Methyl tert-butyl ether |
| TsCl | p-Toluenesulfonyl chloride |
| NaHMDS | Sodium bis(trimethylsilyl)amide |
| NBS | N-Bromosuccinimide |
| Nm | Nanometers |
| NMP | N-Methyl-2-pyrrolidone |
| NMR | Nuclear Magnetic Resonance |
| N-XantPhos | 4,6-Bis(diphenylphosphino)-10H-phenoxazine |
| Pd/C | Palladium on Carbon |
| Prep SFC | Preparative Super Critical Fluid (CO2) |
| p-TsOH | 4-Methylbenzenesulfonic acid |
| RT | Room temperature |
| sat. | Saturated |
| TBAF | Tetrabutylammonium fluoride |
| TBME | Methyl tert-butyl ether |
| TEA | Triethanolamine |
| TFA | Trifluoroacetic acid |
| TFE | 2,2,2-Trifluoroethanol |
| Tf₂O | Trifluoromethanesulfonic anhydride |
| THF | Tetrahydrofuran |
| TLC | Thin Layer Chromatography |
| TMS | Trimethylsilyl |
| PE | Petroleum ether |
| 2-Me-THF | 2-Methyltetrahydrofuran |
| SEMCl | 2-(Trimethylsilyl)ethoxymethyl chloride |
| T₃P | Propanephosphonic acid anhydride |

### EXAMPLES

The following examples serve only to illustrate the invention and its practice. The examples are not to be construed as limitations on the scope or spirit of the invention. In these examples, all temperatures are degrees Celsius unless otherwise noted, and “room temperature” refers to a temperature in a range of from about 20° C. to about 25° C. Reactions sensitive to moisture or air were performed under nitrogen or argon using anhydrous solvents and reagents. Reactions performed using microwave irradiation were normally carried out using an Initiator manufactured by Biotage. Concentration of solutions was carried out on a rotary evaporator under reduced pressure. The progress of reactions was determined by either analytical thin layer chromatography (TLC) usually performed with Merck KGaA glass-backed TLC plates, silica gel 60 F₂₅₄ or liquid chromatography-mass spectrometry (LCMS).

Typically the analytical LCMS system used consisted of a Waters SQD single quadrupole mass spectrometer with electrospray ionization in positive ion detection mode and a Waters Acquity UPLC system (Binary Solvent Manager, Sample Manager, and TUV). The column used was a Waters Aqcuity BEH C18 1×50 mm, 1.7 μm, heated to 50° C. The mobile phase consisted of 0.1% trifluoroacetic acid in water for solvent A and 100% acetonitrile for solvent B. A two-minute run was established at a flow rate of 0.3 ml/min with Initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode.

Preparative HPLC purifications were commonly performed using a Waters XBridge C18, Waters SunFire™ C18 OBD™, Boston Green ODS, or Phenomenex Luna Prep C18 column. The mobile phases consisted of mixtures of acetonitrile (0-100%) in water containing 0.1% TFA and the UV detection range was 210-400 nm. Mobile phase gradients were optimized for the individual compounds. Flash chromatography was usually performed using an ISCO CombiFlash® Rf apparatus, on silica gel (60 Å pore size) in pre-packed RediSep Rf, RediSep Rf Gold, or SepaFlash® columns of the size noted. ¹H NMR spectra were acquired at 500 MHz spectrometers in CDCl₃ solutions unless otherwise noted. Chemical shifts are reported in parts per million (ppm). The residual CHCl₃ peak or tetramethylsilane (TMS) was used as internal reference in CDCl₃ solutions, and the residual CH₃OH peak or TMS was used as internal reference in CD₃OD solutions. Coupling constants (J) are reported in hertz (Hz).

Chiral analytical chromatography was most commonly performed under supercritical fluid chromatography conditions on one of CHIRALPAK® AS, CHIRALPAK® AD, CHIRALCEL® OD, CHIRALCEL® IA, or CHIRALCEL® OJ columns (250×4.6 mm) (Daicel Chemical Industries, Ltd.) with noted percentage of methanol containing 0.05% diethylamine in carbon dioxide, ethanol containing 0.05% diethylamine in carbon dioxide, or ethanol containing 0.05% diethylamine in carbon dioxide as isocratic solvent systems. Chiral preparative supercritical fluid chromatography was commonly conducted on one of CHIRALPAK® AS, CHIRALPAK® AD, CHIRALCEL® OD, CHIRALCEL® IA, CHIRALCEL® OJ columns (30×250 mm or 50×250 mm, Daicel Chemical Industries, Ltd.) or Phenomenex-Amylose-1 (30×250 mm) with noted percentage of methanol containing 0.1% NH₃H₂O in carbon dioxide, ethanol containing 0.10% NH₃H₂O in carbon dioxide, or ethanol containing 0.1% NH₃H₂O in carbon dioxide as isocratic solvent systems.

Catalysts are used in the following procedures. “Grubbs II” is also known as “Grubbs catalyst 2^{nd} generation” and (1,3-Bis(2,4,6-trimethylphenyl)-2-imidazolidinylidene)dichloro(phenylmethylene)(tricyclohexylphosphine)ruthenium; Zhan Catalyst-1B is also known as 1,3-Bis(2,4,6-trimethylphenyl)-4,5-dihydroimidazol-2-ylidene[2-(i-propoxy)-5-(N,N-dimethylaminosulfonyl)phenyl]methyleneruthenium (II) dichloride; Mn(TMHD)₃ is also known as Tris(2,2,6,6-tetramethyl-3,5-heptanedionato)manganese(III); all of which are available from Millipore Sigma.

### Example 1

### Preparation of intermediate Int-1b

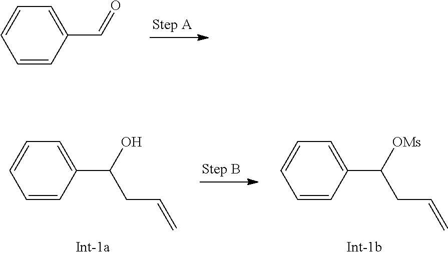

### Step A—Synthesis of Compound Int-1a

To a solution of benzaldehyde (5 g, 47.1 mmol) in THF (50 mL) was added allylmagnesium bromide (141 mL, 141 mmol) at 0° C. The mixture was stirred at 25° C. for 4 h. TLC showed the reaction was completed. The mixture was quenched with aq. NH₄Cl (25 mL) and extracted with EtOAc (50 mL×2). The combined organic solution was washed with brine (30 mL), dried over anhydrous Na₂SO₄, filtered, and concentrated. The residue was purified by column chromatography (SiO₂, 10:1 EtOAc:pet. ether) to afford Int-1a. ¹H NMR (400 MHz, CDCl₃) δ: 7.26-7.35 (m, 5H), 5.77-5.85 (m, 1H), 5.12-5.18 (m, 2H), 4.72 (d, J=8 Hz, 1H), 2.48-2.52 (m, 2H), 2.16 (br s, 1H).

### Step B—Synthesis of Compound Int-1b

To a solution of Int-1a (500 mg, 3.37 mmol) in DCM (10 mL) was added TEA (0.940 mL, 6.75 mmol) and methanesulfonyl chloride (0.394 mL, 5.06 mmol) at 0° C. The mixture was stirred at 0° C. for 1 h, TLC showed the reaction was complete. The mixture was quenched with water (10 mL) and extracted with MTBE (10 mL×3). The organic phase was dried over Na₂SO₄, filtered, and concentrated to afford Int-1b, which was used in the next step without further purification.

### Example 2

### Preparation of Compound 1

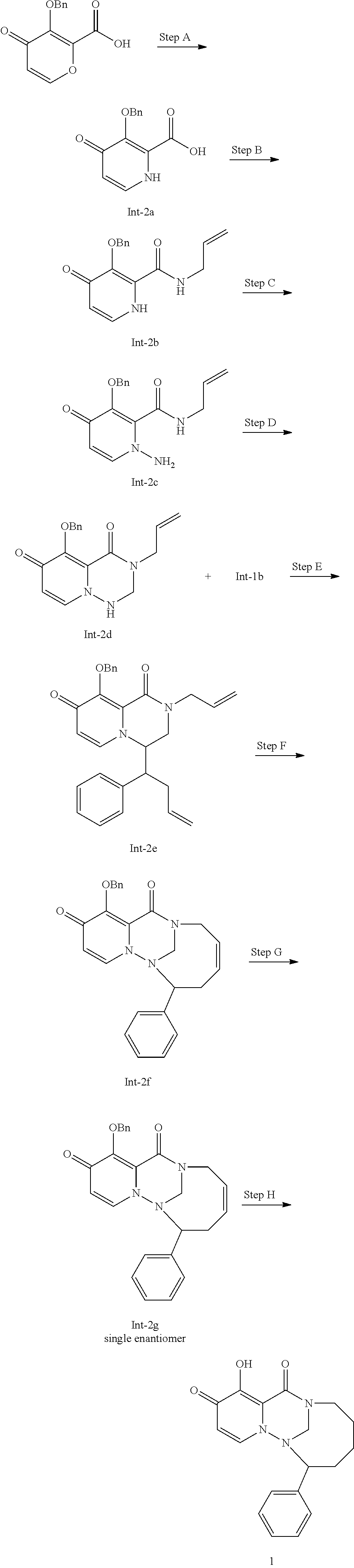

### Step A—Synthesis of Compound Int-2a

A solution of 3-(benzyloxy)-4-oxo-4H-pyran-2-carboxylic acid (5 g, 20.31 mmol) in ammonium hydroxide (40 mL, 20.31 mmol) was stirred at 25° C. for 16 h. LCMS showed the reaction was complete. The mixture was directly concentrated in vacuo, then to the mixture was added water (10 mL). HCl (2 M) was added to adjust pH=2. The mixture was filtered to give Int-2a. Mass calculated for C₁₃H₁₁NO₄: 245.1; found: 246.0 (M+H)⁺.

### Step B—Synthesis of Compound Int-2b

To a solution of Int-2a (500 mg, 2.039 mmol) in DMF (20 mL) was added HATU (1163 mg, 3.06 mmol) and DIPEA (1.424 mL, 8.16 mmol). The mixture was stirred at 0° C. for 30 min. Then allylamine hydrochloride (283 mg, 3.06 mmol) was added and the reaction was stirred at 0° C. for 2 hours. LCMS showed the reaction was complete. The mixture was quenched with water (30 mL) and extracted with DCM (30 mL×3). The organic solution was combined, washed with brine (30 mL×2), dried over anhydrous Na₂SO₄, filtered, and concentrated. The residue was purified by flash silica gel chromatography (ISCO®, 12 g SepaFlash® Silica Flash Column, 0-10% MeOH/DCM gradient, 30 min, dry load) to afford Int-2b. Mass calculated for C₁₆H₁₆N₂O₃: 284.1; found: 285.1 (M+H)⁺.

### Step C—Synthesis of Compound Int-2c

To a solution of Int-2b (400 mg, 1.407 mmol) in DMF (25 mL) was added K₂CO₃ (389 mg, 2.81 mmol) and o-(2,4-dinitrophenyl)hydroxylamine (420 mg, 2.110 mmol) at 25° C. The solution was stirred at 25° C. for 4 h. LCMS showed the reaction was complete. The mixture was filtered, concentrated, and purified by flash silica gel chromatography (ISCO®, 4 g SepaFlash® Silica Flash Column, 0-10% MeOH/DCM gradient, 30 min, dry load) to give Int-2c. Mass calculated for C₁₆H₁₇N₃O₃: 299.1; found: 300.1 (M+H)⁺.

### Step D—Synthesis of Compound Int-2d

To a solution of Int-2c (250 mg, 0.835 mmol) and paraformaldehyde (37.6 mg, 1.253 mmol) in DMF (3 mL) was added acetic acid (0.3 mL). The solution was stirred at 120° C. for 4 h. TLC showed the reaction was complete. The mixture was filtered, concentrated, and purified by flash silica gel chromatography (ISCO®, 4 g SepaFlash® Silica Flash Column, 0-10% MeOH/DCM gradient, 30 min, dry load) to afford Int-2d. Mass calculated for C₁₇H₁₇N₃O₃: 311.1; Found: 312.1 (M+H)⁺.

### Step E—Synthesis of Compound Int-2e

To a solution of Int-2d in DMF (1 mL) was added NaH (70.7 mg, 1.768 mmol, 10%) and Int-1b (200 mg, 0.884 mmol) at 0° C. The solution was stirred at 0° C. for 1 h. LCMS showed the reaction was complete. The mixture was quenched with water (30 mL) and extracted with DCM (30 mL×3). The organic solution was combined, washed with brine (30 mL×2), dried with anhydrous Na₂SO₄, filtered, and concentrated. The crude product was purified by preparative HPLC (Boston Green ODS 30×150 mm 5 μm, 50-70% water (0.1% TFA)/MeCN gradient over 10 min, 100% MeCN hold for 2 min, 25 mL/min, 7 injections) to give Int-2e. Mass calculated for C₂₇H₂₇N₃O₃: 441.2; found: 442.2 (M+H)⁺.

### Step F—Synthesis of Compound Int-2f

To a solution of Int-2e (700 mg, 1.585 mmol) in DCE (15 mL) was added Grubbs 2^{nd} generation catalyst (673 mg, 0.793 mmol). The solution was stirred at 50° C. for 18 h. LCMS showed the reaction was complete. Then the reaction mixture was filtered and concentrated. The crude product was purified by preparative HPLC (Boston Green ODS 30×150 mm, 5 μm, 28-58% water (0.1% TFA)/MeCN gradient over 10 min, 100% MeCN hold for 2 min, 25 mL/min, 12 injections) to give Int-2f as a ˜1:2 mixture of isomers. Mass calculated for C₂₅H₂₃N₃O₃: 413.2; found: 414.2 (M+H)⁺.

### Step G—Preparation of Compound Int-2g

Int-2f was purified by preparative chiral SFC (Phenomenex-Amylose-1 (250 mm×30 mm, 5 μm), 35% EtOH (containing 0.1% NH₃H₂O) in CO₂, 60 mL/min, 80 injections) to afford Int-2g (single enantiomer). Mass calculated for C₂₅H₂₃N₃O₃: 413.2; Found: 414.2 (M+H)⁺.

### Step H—Synthesis of Compound 1

To a solution of Int-2g (25 mg, 0.063 mmol) in MeOH (10 mL) was added palladium on carbon (19.98 mg, 0.019 mmol). The reaction mixture was degassed under vacuum and purged with H₂ for 3 times, then stirred at 25° C. for 15 mins under a H₂ balloon. LCMS showed the reaction was complete. The residue was diluted with MeOH (3 mL) and filtered. The crude product was purified by preparative HPLC (YMC-Actus Pro C18 30×150 mm, 5 μm, 30-60% water (0.10% TFA)/MeCN over 11 min, 100% MeCN hold for 1.1 min, 40 mL/min, 2 injections) to afford 1. ¹H NMR (400 MHz, CDCl₃) δ: 7.32-7.38 (m, 3H), 7.11-7.29 (m, 2H), 6.32 (s, 1H), 6.03 (s, 1H), 5.18-5.31 (m, 2H), 4.72-4.74 (m, 1H), 4.32-4.36 (m, 1H), 3.12-3.16 (m, 1H), 2.17-2.26 (m, 3H), 2.10-2.17 (m, 1H), 1.82-1.85 (m, 2H). Mass calculated for C₁₈H₁₉N₃O₃: 325.1; found: 326.2 (M+H)⁺.

### Example 3

### Preparation of Compounds 2-5

Using procedures similar to those described in Examples 1 and 2 and appropriate starting materials, the following compounds were prepared:

| Compound | | | |
|---|---|---|---|
| # | Structure | ¹H NMR | MS |
| 2 | 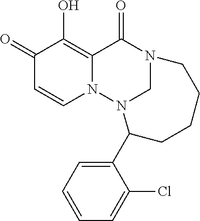 [SMILES: O=C1c2c(O)c(=O)ccn2N2CN1CCCCC2c1ccccc1Cl] | (500 MHz, CDCl₃) δ: 7.35-7.52 (m, 1H), 7.31-7.34 (m, 1H), 7.12-7.16 (m, 1H), 6.83-6.85 (m, 1H), 6.33-6.37 (m, 1H), 6.01-6.05 (m, 1H), 5.22-5.32 (m, 2H), 5.09-5.11 (m, 1H), 4.30-4.34 (m, 1H), 3.13-3.17 (m, 1H), 2.14-2.24 (m, 4H), 1.81-1.85 (m, 2H) | Mass calculated for C₁₈H₁₈ClN₃O₃: 359.1; found: 360.0 (M + H)⁺ |
| 3 | 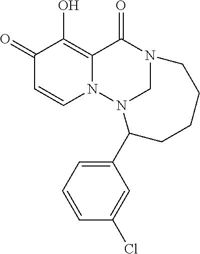 [SMILES: O=C1c2c(O)c(=O)ccn2N2CN1CCCCC2c1cccc(Cl)c1] | (500 MHz, CDCl₃) δ: 7.32-7.34 (s, 1H), 7.23-7.26 (m, 1H), 7.13-7.21 (m, 1H), 6.80 (s, 1H), 6.16-6.20 (d, J =7.2 Hz, 2H), 5.28-5.31 (d, 1H), 4.79-4.82 (d, 1H), 4.42-4.50 (m, 2H), 2.99-3.02 (m, 1H), 2.22-2.30 (m, 3H), 2.05 (s, 1H), 1.83-1.86 (m, 1H), 1.61-1.63 (m, 1H) | Mass calculated for C₁₈H₁₈ClN₃O₃: 359.1; found: 360.0 (M + H)⁺ |
| 4 | 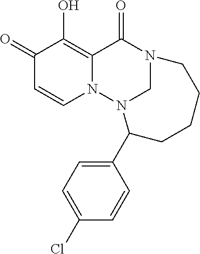 [SMILES: O=C1c2c(O)c(=O)ccn2N2CN1CCCCC2c1ccc(Cl)cc1] | (400 MHZ, CD₃OD) δ 7.34 (d, J =8.31 Hz, 2H), 7.12 (br d, J =7.83 Hz, 2H), 6.47 (d, J-7.58 Hz, 1H), 6.11 (d, J =7.58 Hz, 1H), 5.26-5.34 (m, 1H), 5.15-5.24 (m, 1H), 4.73 (br d, J =10.27 Hz, 1H), 4.33 (br d, J =11.74 Hz, 1H), 3.09-3.20 (m, 1H), 2.02-2.24 (m, 4H), 1.77-1.87 (m, 2H) | Mass calculated for C₁₈H₁₈ClN₃O₃: 359.1; found: 360.0 (M + H)⁺ |
| 5 | 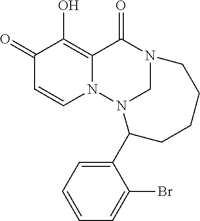 [SMILES: O=C1c2c(O)c(=O)ccn2N2CN1CCCCC2c1ccccc1Br] | (400 MHZ, CDCl₃) δ 7.64-7.66 (br s, 1H), 7.17-7.11 (m,2H), 6.69 (s, 1H), 6.19 (br s, 1H), 6.09-6.18 (m, 1H), 5.25-5.28 (m, 1H), 4.79-4.90 (m, 2H), 4.43-4.48 (m, 1H), 2.98-3.01 (m, 1H), 2.10-2.20 (m, 3H), 1.80-1.83 (m, 1H), 1.64-1.67 (m, 1H) | Mass calculated for C₁₈H₁₈BrN₃O₃: 403.1, 405.1; found: 404.1, 406.1 (M + H)⁺. |

### Example 4

### Preparation of Compounds 6-7

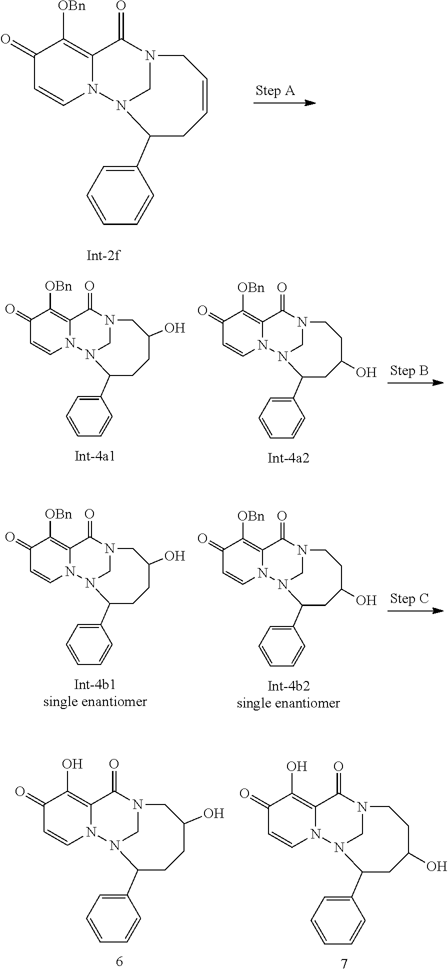

### Step A Synthesis of Compounds Int-4a and Int-4b

To a solution of Int-2f (100 mg, 0.242 mmol) in 2-propanol (30 mL) were added phenylsilane (0.090 ml, 0.726 mmol) and Mn(TMHD)₃ (29.3 mg, 0.048 mmol) at room temperature. The reaction was evacuated and backfilled with 02 three times and the mixture was vigorously stirred at 25° C. After 20 min, the reaction mixture was again evacuated and backfilled with 02 three times and stirred at 25° C. for 1 h. LCMS showed the reaction was complete. The reaction mixture was diluted with DCM (60 mL), washed with brine (20 mL), dried over Na₂SO₄, filtered, and concentrated. The crude product was purified by flash silica gel chromatography (ISCO®; 12 g SepaFlash® Silica Flash Column, Eluent of 0˜5% DCM/MeOH gradient, 20 min, dry load) to afford a 1:1 mixture of compounds Int-4a1 and Int-4a2. Mass calculated for C₂₅H₂₅N₃O₄: 431.2; found: 432.1 (M+H)⁺.

### Step B—Preparation of Compounds Int-4b1 and Int-4b2

The mixture of Int-4a1 and Int-4a2 was purified by preparative chiral SFC (DAICEL CHIRALCEL OJ, 250 mm×50 mm, 10 μm, 40% MeOH (containing 0.1% NH₃H₂O) in CO₂, 200 mL/min) to afford Int-4b1 and Int-4b2. Mass calculated for C₂₅H₂₅N₃O₄: 431.2; found for Int-4b1: 432.2 (M+H)⁺; found for Int-4b2: 432.2 (M+H)⁺.

### Step C—Synthesis of Compounds 6 and 7

To a solution of Int-4b1 (20 mg, 0.046 mmol) in MeCN (5 mL) was added magnesium bromide (42.7 mg, 0.232 mmol) at 25° C. and the mixture was stirred for 16 h. LCMS showed the starting material had disappeared and the desired product was detected. The mixture was diluted with MeOH (0.2 mL) and then purified by reverse-phase HPLC on (Boston Green ODS, 30×150 mm, 5 μm, 20-50% water (0.1% TFA)/MeCN gradient over 10 min, 100% MeCN hold for 1 min, 25 mL/min, 220 nm detection) followed by lyophilization to give 6. ¹H NMR (400 MHz, CD₃OD): δ 7.17-7.57 (m, 3H), 7.09 (br s, 2H), 6.35 (br d, J=7.3 Hz, 1H), 5.92-6.13 (m, 1H), 5.14-5.45 (m, 2H), 4.71-4.78 (m, 1H), 4.38-4.57 (m, 1H), 4.18 (br s, 1H), 2.89 (dd, J=13.1, 10.4 Hz, 1H), 1.81-2.38 (m, 4H). Mass calculated for C₁₈H₁₉N₃O₄: 341.1; found: 342.1 (M+H)⁺.

In like manner Int-4b2 afforded 7; ¹H NMR (400 MHz, CD₃OD): δ 7.27-7.46 (m, 3H), 7.13 (br s, 2H), 6.32 (d, J=7.3 Hz, 1H), 6.01 (d, J=7.6 Hz, 1H), 5.21 (d, J=14.2 Hz, 1H), 5.01 (d, J=14.2 Hz, 1H), 4.37-4.49 (m, 1H), 4.07-4.24 (m, 1H), 3.17-3.27 (m, 1H), 2.16-2.47 (m, 3H), 2.01 (br d, J=11.7 Hz, 1H). Mass calculated for C₁₈H₁₉N₃O₄: 341.1; found: 342.1 (M+H)⁺.

### Example 5

### Preparation of Compound 8

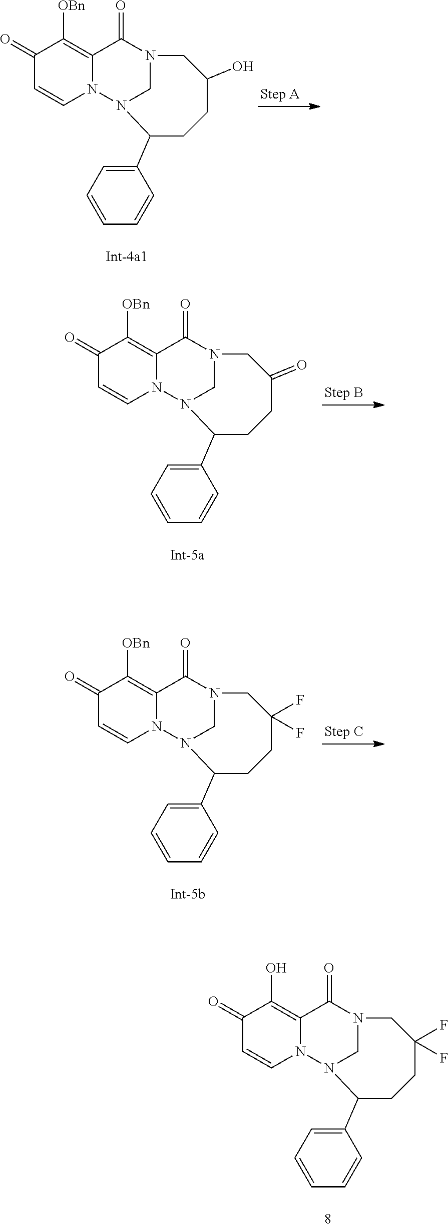 [SMILES: O=C1c2c(OCc3ccccc3)c(=O)ccn2N2CN1CC(O)CCC2c1ccccc1 ; O=C1CCC(c2ccccc2)N2CN(C1)C(=O)c1c(OCc3ccccc3)c(=O)ccn12 ; O=C1c2c(OCc3ccccc3)c(=O)ccn2N2CN1CC(F)(F)CCC2c1ccccc1 ; O=C1c2c(O)c(=O)ccn2N2CN1CC(F)(F)CCC2c1ccccc1]

Step A—Synthesis of Compound Int-5a

To a solution of Int-4a1 (as a 1:1 mixture with Int-4a2, 280 mg, 0.324 mmol) in DCM (5 mL) was added DMP (413 mg, 0.973 mmol) at 0° C. The mixture was warmed to rt and stirred for 12 h. LCMS showed the reaction was completed. The reaction was quenched with water (20 mL) and the aqueous phase was extracted with DCM:MeOH (10:1, 20 mL×3). The organic phase was concentrated and diluted with MeOH (3 mL). The residue was purified by prep-HPLC (Boston Green ODS 30×150 mm, 5 μm, water (0.1% TFA)/MeCN, 30-50% gradient over 10 min, 100% MeCN hold for 2 min, 25 mL/min) to afford Int-5a as a 1:1 mixture with the corresponding regioisomer. Mass calculated for C₂₅H₂₃N₃O₄: 429.2; found: 430.2 (M+H)⁺.

### Step B Synthesis of Compound Int-5b

To a solution of Int-5a (as a 1:1 mixture with its regioisomer, 100 mg, 0.116 mmol) in DCE (5 mL) was added BAST (0.021 mL, 0.116 mmol) at 0° C. The reaction solution was stirred at 55° C. for 12 h. LCMS showed the reaction was completed. The mixture was quenched with sat. aq. NaHCO₃ (10 mL) and the aqueous phase was extracted with DCM (10 mL×2). The combined organic extracts were concentrated and the residue was purified by preparative HPLC (Boston Green ODS 30×150 mm, 5 μm, water (0.1% TFA)/MeCN, 38-68% gradient over 10 min, 100% MeCN hold for 2 min, 25 mL/min) to give a 1:1 mixture of product regioisomers. This material was further purified by preparative SFC (DAICEL CHIRALPAK AD, 250 mm×30 mm, 10 μm, 30% EtOH (containing 0.1% NH₃H₂O) in CO₂, 70 mL/min) to afford Int-5b. Mass calculated for C₂₅H₂₃F₂N₃O₃: 451.2; found: 452.2 (M+H)⁺.

### Step C—Synthesis of Compound 8

To a solution Int-5b (3 mg, 6.65 μmol) in MeCN (3 mL) was added magnesium bromide (12.23 mg, 0.066 mmol). The mixture was stirred at 30° C. for 12 h. LCMS showed the reaction was complete. The mixture was concentrated in vacuo, diluted with MeOH (3 mL), and filtered. The filtrate was purified by preparative HPLC (Boston Green ODS 30×150 mm, 5 μm, water (0.1% TFA)/MeCN, 30-60% gradient over 10 min, 100% MeCN hold for 2 min, 25 mL/min) to afford 8. ¹H NMR (400 MHz, CDCl₃) δ: 7.34-7.39 (m, 3H), 7.12-7.14 (m, 2H), 6.64 (d, J=7.43 Hz, 1H), 6.28 (d, J=7.43 Hz, 1H), 5.12-5.15 (m, 1H), 5.00-5.04 (m, 1H), 4.75-4.90 (m, 1H), 3.82 (d, J=9.39 Hz, 1H), 3.55 (br t, J=13.69 Hz, 1H), 2.36-2.54 (m, 2H), 2.04-2.31 (m, 2H). Mass calculated for C₁₈H₁₇F₂N₃O₃: 361.1; found: 362.2 (M+H)⁺.

### Example 6

### Preparation of Compound 9

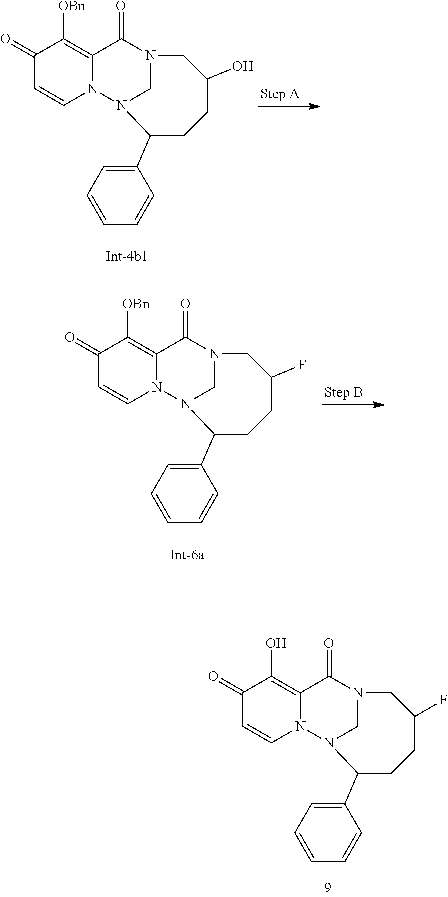 [SMILES: O=C1c2c(OCc3ccccc3)c(=O)ccn2N2CN1CC(O)CCC2c1ccccc1 ; O=C1c2c(OCc3ccccc3)c(=O)ccn2N2CN1CC(F)CCC2c1ccccc1 ; O=C1c2c(O)c(=O)ccn2N2CN1CC(F)CCC2c1ccccc1]

### Step A—Synthesis of Compound Int-6a

To a solution of Int-4b1 (20 mg, 0.046 mmol) in DCM (2 mL) was added DAST (0.031 mL, 0.232 mmol) at 0° C. The reaction solution was stirred at 0° C. for 20 mins. TLC showed the starting material was consumed and desired product was detected. The reaction solution was directly purified by preparative TLC (10% MeOH in DCM) to afford Int-6a. Mass calculated for C₂₅H₂₄FN₃O₃: 433.2; found: 434.1 (M+H)⁺.

### Step B—Synthesis of Compound 9

To a solution of Int-6a (12 mg, 0.014 mmol) in MeCN (4 mL) was added magnesium bromide (2.61 mg, 0.014 mmol) at 25° C. and stirred for 16 h. LCMS showed the starting material had disappeared and the desired product was detected. The mixture was diluted with MeOH (0.2 mL) and then purified by reverse phase HPLC on Boston Green ODS 150×30 mm, 5 μm, water (0.1% TFA)/MeCN eluent, 30-50% gradient over 10 min, 100% MeCN hold for 1 min, 25 mL/min, detection at 220 nm) followed by lyophilization to give 9. ¹H NMR (CD₃OD, 500 MHz): δ7.29-7.54 (m, 3H), 7.15 (br s, 2H), 6.36 (d, J=7.3 Hz, 1H), 5.99 (br d, J=7.6 Hz, 1H), 5.22-5.41 (m, 2H), 5.00-5.14 (m, 1H), 4.79 (br d, J=9.9 Hz, 1H), 4.63 (br d, J=12.2 Hz, 1H), 3.08-3.27 (m, 1H), 2.43-2.57 (m, 1H), 2.08-2.36 (m, 3H). Mass calculated for C₁₈H₁₈FN₃O₃: 343.1; found: 344.1 (M+H)⁺.

### Example 7

### Preparation of Compound 10

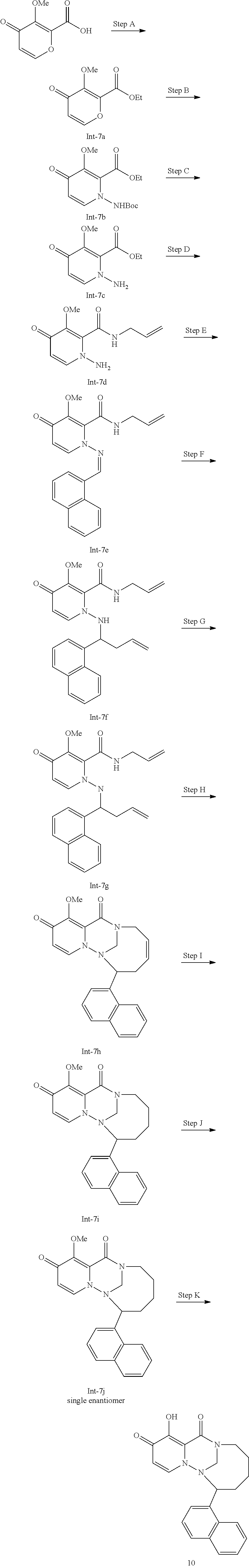

### Step A—Synthesis of Compound Int-7a

Into a 10 L 4-necked round-bottom flask purged and maintained with an inert atmosphere of nitrogen was placed 3-methoxy-4-oxopyran-2-carboxylic acid (690 g, 4055 mmol, 1.00 equiv), dimethylformamide (10 L), K₂CO₃ (903.43 g, 6489 mmol, 1.60 equiv), and ethyl iodide (1202.g, 7706 mmol, 1.90 equiv). The resulting solution was stirred for overnight at 25° C. The resulting solution was diluted with 15 L of EtOAc. The resulting mixture was washed with 4×7 L of H₂O and 3×5 L of brine. The mixture was dried over anhydrous sodium sulfate. The solids were filtered out and the filtrate was concentrated under vacuum. The residue was applied to a silica gel column with EtOAc:pet. ether (gradient 1:30 to 1:5) to afford Int-7a. Mass calculated for C₉H₁₀O₅: 198.1; found: 199.0 (M+H)⁺.

### Step B—Synthesis of Compound Int-7b

A 500 mL round-bottom flask equipped with a magnetic stirring bar was charged with Int-7a (20 g, 101 mmol), DMA (168 ml), tert-butyl hydrazinecarboxylate (14.67 g, 111 mmol), and pyridinium p-toluenesulfonate (76 g, 303 mmol). The mixture was heated to 60° C. with stirring for 17 h. LCMS showed the reaction was near complete. The mixture was diluted with EtOAc and washed once with sat. aq. NH₄Cl and the layers were separated. The aqueous layer was extracted with one additional portion of EtOAc. The combined organic layers were washed with water and brine, dried over Na₂SO₄, filtered, and concentrated in vacuo. The residue was purified by flash chromatography on silica gel (ISCO, 330 g cartridge) eluting with 0-55% (3:1 EtOAc:EtOH)/hexanes to afford Int-7b. Mass calculated for C₁₄H₂₀N₂O₆: 312.1; found: 313.3 (M+H)⁺.

### Step C—Synthesis of Compound Int-7c

To a 250 mL round-bottom flask containing Int-7b (7.6 g, 24.33 mmol) were added DCM (122 ml) and trifluoroacetic acid (37.2 ml, 487 mmol) and the mixture was stirred at rt. After 1.25 h the mixture was concentrated in vacuo. The residue was purified by flash chromatography on silica gel (ISCO, 330 g cartridge) eluting with 0-10% MeOH/DCM (fast gradient, then isocratic) to afford Int-7c. Mass calculated for C₉H₁₂N₂O₄: 212.1; found: 213.1 (M+H)⁺.

### Step D—Synthesis of Compound Int-7d

A 100 mL round-bottom flask equipped with a magnetic stirring bar was charged with Int-7c (1.00 g, 4.71 mmol) and ethanol (23.56 ml). Allylamine (7.05 ml, 94 mmol) was added and the mixture was heated to 80° C. with stirring. After 19 h, the mixture was concentrated in vacuo to afford Int-7d. Mass calculated for C₁₀H₁₃N₃O₃: 223.1; found: 224.1 (M+H)⁺.

### Step E—Synthesis of Compound Int-7e

A suspension of Int-7d (658 mg, 2.95 mmol), 1-naphthaldehyde (400 μl, 2.95 mmol) and TFA (681 μl, 8.84 mmol) in dioxane (7369 μl) was heated to 100° C. with stirring for 1 h. LCMS showed complete conversion to desired product. The mixture was cooled to rt and concentrated in vacuo. The crude material was purified by gradient elution on silica gel (RediSep-Rf-40 g, 0-20% MeOH/DCM, 30 minute gradient) to afford Int-7e. Mass calculated for C₂₁H₁₉N₃O₃: 361.1; found: 362.4 (M+H)⁺.

### Step F—Synthesis of Compound Int-7f

A solution of Int-7e (710 mg, 1.965 mmol) in THF (10 mL) was cooled to 0° C. and was treated with allylmagnesium chloride (2456 μl, 4.91 mmol) at 0° C. and stirred for 2 min. LCMS showed good, clean conversion to desired product. The mixture was quenched (after stirring for 5 min at 0° C. total) with sat. aq. NH₄Cl, and then partitioned between 1:1 sat. aq. NH₄Cl:water and EtOAc. The aqueous phase was back-extracted with EtOAc. The combined organic phases were dried over Na₂SO₄, filtered and concentrated. The crude material was purified by gradient elution on silica gel (RediSep-Rf-40 g, 0-10% MeOH/DCM, 20 minute gradient) to afford Int-7f. Mass calculated for C₂₄H₂₅N₃O₃: 403.2; found: 404.4 (M+H)⁺.

### Step G—Synthesis of Compound Int-7g

A solution of Int-7f in DCE (12.500 mL) in a 20 mL microwave vial was treated with paraformaldehyde (561 mg, 18.70 mmol) and 4-methylbenzenesulfonic acid hydrate (23.71 mg, 0.125 mmol), sealed, and heated to 150° C. in a microwave reactor with stirring for 1.5 h. The mixture was cooled to rt and LCMS showed complete conversion to desired product. The mixture was partitioned between water and DCM. The aqueous phase was back-extracted with DCM. The combined organic phases were dried over Na₂SO₄, filtered and concentrated. The crude material was purified by gradient elution on silica gel (RediSep-Rf-40 g, 0-10% MeOH/DCM, 20 minute gradient) to afford Int-7g. Mass calculated for C₂₅H₂₅N₃O₃: 415.2; found: 416.4 (M+H)⁺.

### Step H—Synthesis of Compound Int-7h

A solution of Int-7g (296 mg, 0.712 mmol) in DCE (142 mL) was degassed for 5 min by bubbling N₂ gas through the solution. The solution was treated with Grubbs 2^{nd} generation catalyst (121 mg, 0.142 mmol) and stirred at 60° C. for 1 h. LCMS showed good, clean conversion to desired product. The mixture was cooled to rt and concentrated in vacuo. The crude material was purified by gradient elution on silica gel (RediSep-Rf-40 g, 0-10% MeOH/DCM, 20 minute gradient) to afford Int-7h. Mass calculated for C₂₃H₂₁N₃O₃: 387.2; found: 388.4 (M+H)⁺.

### Step I—Synthesis of Compound Int-7i

A solution of Int-7h (263.7 mg, 0.681 mmol) in MeOH/EtOAc (3.4/3.4 mL) was treated with palladium hydroxide (20 wt %) (96 mg, 0.136 mmol). The flask was filled with H_{2(g)} and purged vigorously for 10 seconds. The solution was stirred under an H_{2(g)} atmosphere (balloon) at rt for 2 h. LCMS showed that the reaction was complete. The catalyst was filtered off through a syringe filter, washing with MeOH and EtOAc. The filtrate was concentrated. The crude material was purified by gradient elution on reverse phase (Phenomenex Luna Prep C18, 50×250 mm, 5 μm, MeCN/water with 0.10% TFA modifier, 20-65% gradient over 30 min, 90 mL/min). Pure fractions were combined and concentrated in vacuo azeotroping with MeCN to yield Int-7i as a mixture of product isomers. Mass calculated for C₂₃H₂₃N₃O₃: 389.2; found: 390.4 (M+H)⁺.

### Step J—Preparation of Compound 7j

Int-7i was purified by preparative SFC (AS-H, 2×25 cm, 30% MeOH with 0.10% DEA, 60 mL/min) to afford Int-7j. Mass calculated for C₂₃H₂₃N₃O₃: 389.2; found: 390.3 (M+H)⁺.

### Step K—Synthesis of Compound 10

A solution of Int-7i (40 mg, 0.103 mmol) in DMF (1 mL) was treated with lithium chloride (131 mg, 3.08 mmol) and heated to 100° C. for 2 h. LCMS showed complete conversion to desired product. The crude material was directly purified by gradient elution on reverse-phase HPLC (XBridge C18 30×250 mm, 10 μm, MeCN/water with 0.1% TFA modifier, 5-95% gradient over 20 min, 50 mL/min, 1 injection). Pure fractions were concentrated in vacuo to yield 10. Mass calculated for C₂₂H₂₁N₃O₃: 375.2; found: 376.3 (M+H)⁺.

### Example 8

### Preparation of Compounds 11-16

Using procedures similar to those described in Example 7 and appropriate starting materials, the following compounds were prepared:

| Compound | | | |
|---|---|---|---|
| # | Structure | ¹H NMR | MS |
| 11 | 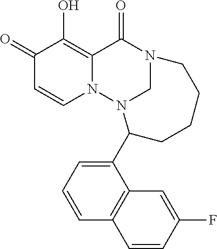 [SMILES: O=C1c2c(O)c(=O)ccn2N2CN1CCCCC2c1cccc2ccc(F)cc12] | N/A | Mass calculated for C₂₂H₂₀FN₃O₃: 393.2; found: 394.4 (M + H)⁺. |
| 12 | 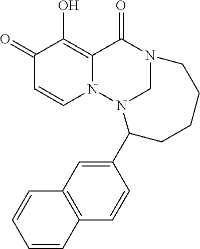 [SMILES: O=C1c2c(O)c(=O)ccn2N2CN1CCCCC2c1ccc2ccccc2c1] | N/A | Mass calculated for C₂₂H₂₁N₃O₃: 375.2; found: 376.3 (M + H)⁺. |
| 13 | 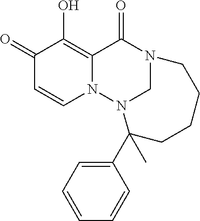 [SMILES: CC1(c2ccccc2)CCCCN2CN1n1ccc(=O)c(O)c1C2=O] | N/A | Mass calculated for C₁₉H₂₁N₃O₃: 339.2; found: 340.3 (M + H)⁺. |
| 14 | 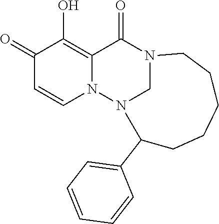 [SMILES: O=C1c2c(O)c(=O)ccn2N2CN1CCCCCC2c1ccccc1] | (500 MHZ, CDCl₃) δ 7.31 (d, J = 7.3 Hz, 3H), 7.09 (d, J = 6.7 Hz, 2H), 6.66 (d, J = 7.1 Hz, 1H), 6.36 (d, J = 7.2 Hz, 1H), 5.19 (d, J = 14.4 Hz, 1H), 5.02 (d, J = 14.3 Hz, 1H), 4.48 (ddd, J = 14.8, 11.2, 4.4 Hz, 1H), 3.85 (s, 1H), 2.99 (dt, J = 14.3, 4.0 Hz, 1H), 2.25-2.15 (m, 2H), 2.08- 1.95 (m, 1H), 1.91-1.76 (m, 2H), 1.75-1.50 (m, 3H). | Mass calculated for C₁₉H₂₁N₃O₃: 339.2; found: 340.3 (M + H)⁺. |
| 15 | 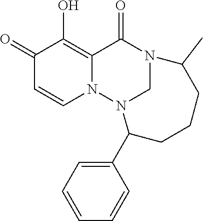 [SMILES: CC1CCCC(c2ccccc2)N2CN1C(=O)c1c(O)c(=O)ccn12] | (500 MHz, CDCl₃) δ 7.40-7.30 (m, 3H), 7.05 (d, J = 6.9 Hz, 2H), 6.35 (d, J = 7.5 Hz, 1H), 6.09 (d, J = 7.5 Hz, 1H), 4.99- 4.88 (m, 2H), 4.78 (q, J = 6.8, 6.4 Hz, 1H), 4.43 (d, J = 8.5 Hz, 1H), 2.25-2.10 (m, 2H), 2.05- 1.90 (m, 3H), 1.85-1.75 (m, 1H), 1.30 (d, J = 6.9 Hz, 3H). | Mass calculated for C₁₉H₂₁N₃O₃: 339.2; found: 340.3 (M + H)⁺. |
| 16 | 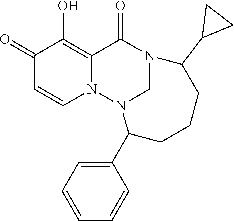 [SMILES: O=C1c2c(O)c(=O)ccn2N2CN1C(C1CC1)CCCC2c1ccccc1] | N/A | Mass calculated for C₂₁H₂₃N₃O₃: 365.2; found: 366.3 (M + H)⁺. |

### Example 9

### Preparation of Compounds 17-20

Starting from intermediates similar in structure to Int-2f or Int-7h and using the general methods for chiral resolution and deprotection as described in Example 7 (steps J and K) the following compounds were prepared:

| Compound | | | |
|---|---|---|---|
| # | Structure | ¹H NMR | MS |
| 17 | 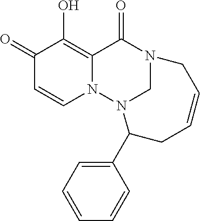 [SMILES: O=C1c2c(O)c(=O)ccn2N2CN1C/C=C\CC2c1ccccc1] | (400 MHZ, CDCl₃) δ: 7.31-7.37 (m, 4H), 7.11 (s, 2H), 6.45- 6.47(m, 1H), 5.92-5.99 (m, 1H), 5.75-5.77 (m, 1H), 5.23-5.28 (m, 1H), 5.01-5.12 (m, 3H), 4.61 (s, 1H), 2.90-2.92 (m, 1H), 2.45- 2.52 (m, 1H) | Mass calculated for C₁₈H₁₇N₃O_{3:} 323.1; found: 324.1 (M + H)⁺. |
| 18 | 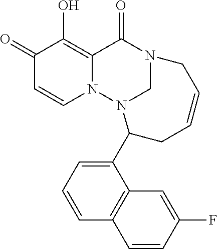 [SMILES: O=C1c2c(O)c(=O)ccn2N2CN1C/C=C\CC2c1cccc2ccc(F)cc12] | N/A | Mass calculated for C₂₂H₁₈FN₃O_{3:} 391.1; found: 392.3 (M + H)⁺. |
| 19 | 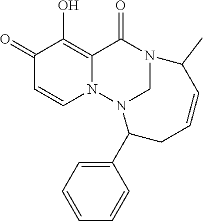 [SMILES: CC1/C=C\CC(c2ccccc2)N2CN1C(=O)c1c(O)c(=O)ccn12] | (500 MHZ, CD₃OD) δ 7.39- 7.28 (m, 3H), 7.12 (s, 2H), 6.54 (d, J = 7.5 Hz, 1H), 6.02 (d, J = 7.5 Hz, 1H), 5.97-5.87 (m, 1H), 5.69 (d, J = 11.5 Hz, 1H), 5.41 (m, J = 7.6 Hz, 1H), 5.18-5.07 (m, 2H), 4.60 (d, J = 10.7 Hz, 1H), 3.01-2.84 (m, 1H), 2.45 (dd, J = 15.2, 9.4 Hz, 1H), 1.37 (d, J = 7.4 Hz, 3H). | Mass calculated for C₁₉H₁₉N₃O_{3:} 337.1; found: 338.3 (M + H)⁺. |
| 20 | 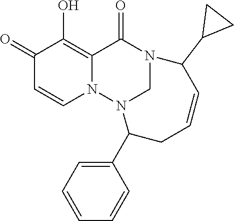 [SMILES: O=C1c2c(O)c(=O)ccn2N2CN1C(C1CC1)/C=C\CC2c1ccccc1] | (500 MHZ, CD₃OD) δ 7.38- 7.28 (m, 3H), 7.12 (s, 2H), 6.47 (d, J = 7.6 Hz, 1H), 6.00-5.84 (m, 3H), 5.26-5.14 (m, 2H), 4.59 (d, J = 10.7 Hz, 1H), 4.47 (dd, J = 9.9, 2.7 Hz, 1H), 2.95- 2.84 (m, 1H), 2.45 (dd, J = 15.2, 9.0 Hz, 1H), 1.15 (dddd, J = 12.6, 10.0, 8.0, 4.7 Hz, 1H), 0.78 (tdd, J = 8.5, 5.6, 3.7 Hz, 1H), 0.55 (m, 3H). | Mass calculated for C₂₁H₂₁N₃O₃: 363.2; found: 364.3 (M + H)⁺. |

### Example 10

### Preparation of Compound 21

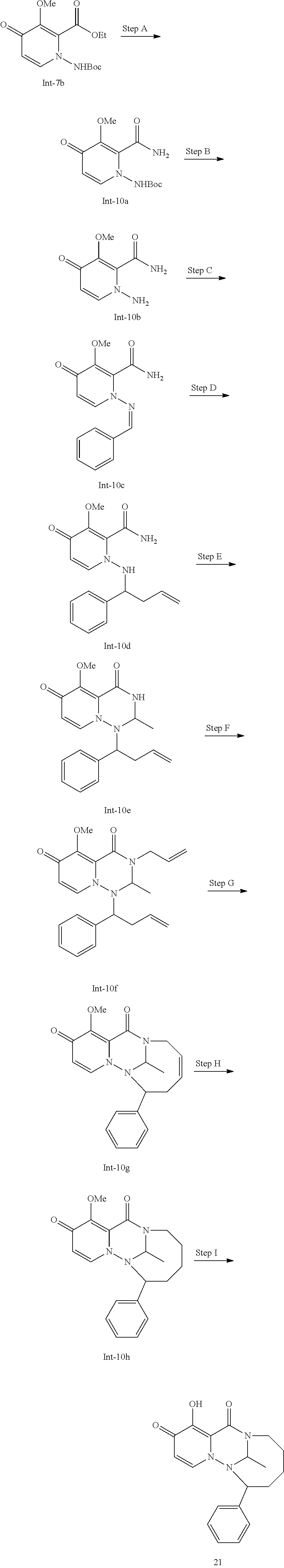

### Step A—Synthesis of Compound Int-10a

A 300 mL stainless steel Parr high-pressure vessel was charged with Int-7b (10 g, 32.0 mmol) as a solution in ethanol (10 ml). The vessel was cooled to −78° C. in an acetone/dry ice bath. Ammonia (20 ml, 924 mmol) was condensed into a collection flask (also at −78° C.), then added to the Parr reactor. The top of the reactor was assembled and sealed before the vessel was removed from the bath and allowed to warm to rt. After checking for any leaks, the vessel was heated to 100° C. behind a blast shield for 20 h. After this time the reaction was cooled to rt and then to −78° C. The vessel was vented and the reaction was judged to be complete by LCMS. The vessel was then warmed to just above rt with warm water to facilitate evaporation of NH₃. The reaction mixture was filtered through paper to remove solids from the reaction, rinsing with MeOH. The solids were found to contain some product by LCMS. The solids were then triturated with DCM/MeOH and filtered. The filtrates were combined and concentrated in vacuo to afford Int-10a. Mass calculated for C₁₂H₁₇N₃O₅: 283.1; found: 284.3 (M+H)⁺.

### Step B—Synthesis of Compound Int-10b

A 500 mL round-bottom flask equipped with a magnetic stirring bar was charged with Int-10a (5.6 g, 19.77 mmol). DCM (99 ml) and trifluoroacetic acid (37.8 ml, 494 mmol) were added and the mixture was stirred at rt. After 1.5 h, the reaction was judged to be complete by LCMS. The mixture was concentrated in vacuo to afford Int-10b. Mass calculated for C₇H₉N₃O₃: 183.1; found: 184.1 (M+H)⁺.

### Step C—Synthesis of Compound Int-10c

A 300 mL round-bottom flask equipped with a magnetic stirring bar was charged with Int-10b (3.62 g, 19.76 mmol), dioxane (49.4 ml), and benzaldehyde (1.815 ml, 17.79 mmol) and heated to 100° C. with stirring for 30 min. LCMS showed complete conversion to desired product. The mixture was cooled to rt and concentrated in vacuo. The material was dissolved in DCM/MeOH. 15 g of loose silica gel was added and the mixture was concentrated to adsorb onto silica. The crude material was purified by gradient elution on silica gel (dry load, RediSep-Rf-120 g, 0-20% MeOH/DCM, 30 minute gradient) to afford Int-10c. Mass calculated for C₁₄H₁₃N₃O₃: 271.1; found: 272.2 (M+H)⁺.

### Step D—Synthesis of Compound Int-10d

A suspension of Int-10c (880 mg, 3.24 mmol) in THF (32 mL) was treated slowly with allylmagnesium chloride (6.49 mL, 12.98 mmol) and stirred at rt for 15 min. LCMS showed that the reaction was nearly complete with a minor impurity. The mixture was quenched with sat. aq. NH₄Cl and partitioned between EtOAc and water. LCMS showed product in both layers. The aqueous phase was back-extracted with DCM twice to remove product. The combined organic phases were dried over Na₂SO₄, filtered, and concentrated. The crude material was purified by gradient elution on reverse phase (50×250 mm (5 um) Phenomenex Luna Prep C18, 5-75% MeCN/water w/0.1% TFA modifier over 20 min @90 mL/min, 1 inj). Pure fractions were combined and partitioned between DCM and sat. aq. NaHCO₃. The aqueous phase was back extracted with DCM twice. The combined organic phases were dried over Na₂SO₄, filtered and concentrated to afford Int-10d. Mass calculated for C₁₇H₁₉N₃O₃: 313.1; found: 314.3 (M+H)⁺.

### Step E—Synthesis of Compound Int-10e

A suspension of Int-10d (426 mg, 1.359 mmol) and 4-methylbenzenesulfonic acid hydrate (129 mg, 0.680 mmol) in EtOH (6797 μl) was treated with acetaldehyde (1526 μl, 27.2 mmol), sealed in a microwave vial and heated conventionally in a heating block to 120° C. with stirring for 7 h. LCMS shows conversion to product mixture (˜1:2 diastereomeric mixture). The mixture was concentrated. The crude material was purified by gradient elution on reverse phase HPLC Phenomenex Luna Prep C18, 50×250 mm, 5 μm, MeCN/water with 0.1% TFA modifier 5-65% gradient over 30 min, 90 mL/min). Pure fractions were combined and concentrated in vacuo azeotroping with MeCN to afford Int-10e. Mass calculated for C₁₉H₂₁N₃O₃: 339.2; found: 340.3 (M+H)⁺.

### Step F—Synthesis of Compound Int-10f

A solution of Int-10e (32.3 mg, 0.095 mmol) in DMF (0.5 ml) was treated with allyl bromide (12.35 μl, 0.143 mmol) and Cs₂CO₃ (78 mg, 0.238 mmol). The reaction mixture was stirred at 60° C. for 40 min. LCMS showed complete conversion to desired product. The mixture was partitioned between water and DCM. The aqueous phase was back-extracted with DCM. The combined organic phases were dried over Na₂SO₄, filtered, and concentrated in vacuo. The crude material was purified by gradient elution on silica gel (RediSep-Rf-4 g, 0-10% MeOH/DCM, 12 minute gradient) to afford Int-10f. Mass calculated for C₂₂H₂₅N₃O₃: 379.2; found: 380.4 (M+H)⁺.

### Step G—Synthesis of Compound Int-10′

A solution of Int-10f (26 mg, 0.069 mmol) in DCE (3 mL) was degassed for 2 min by bubbling N₂ gas through the solution. The solution was treated with Zhan Catalyst-1B (10.06 mg, 0.014 mmol) and stirred at 80° C. for 1.5 h. LCMS showed nearly complete conversion to desired product. The crude reaction mixture was concentrated, diluted with a small amount of 1:1 MeCN:water and purified directly by gradient elution on reverse phase (Waters Sunfire OBD C18, 30×150 mm, 5 μm, MeCN/water w/0.1% TFA modifier, 15-85% gradient over 20 min, 40 mL/min). Pure fractions were concentrated in vacuo to afford Int-10g. Mass calculated for C₂₀H₂₁N₃O₃: 351.2; found: 352.4 (M+H)⁺.

### Step H—Synthesis of Compound Int-10h

A solution of Int-10g (30 mg, 0.085 mmol) in 1:1 MeOH:EtOAc (2 mL) was treated with palladium hydroxide (20 wt %) (11.99 mg, 0.017 mmol). The flask was filled with H_{2(g)} and purged vigorously for 10 seconds. The solution was stirred under a H_{2(g)} atmosphere (balloon) at rt for 10 min. LCMS showed that the reaction was complete. The catalyst was filtered off through syringe filter washing with MeOH and EtOAc. The filtrate was concentrated. The crude material was purified by gradient elution on silica gel (RediSep-Rf-4 g, 0-10% MeOH/DCM, 12 minute gradient) to afford a mixture of product isomers. This material was further purified by preparative SFC (AS-H, 2×25 cm, 30% isopropanol, 70 mL/min) to afford Int-10h. Mass calculated for C₂₀H₂₃N₃O₃: 353.2; found: 354.4 (M+H)⁺.

### Step I—Synthesis of Compound 21

A solution of Int-10h (6 mg, 0.017 mmol) in DMF (1 mL) was treated with lithium chloride (21.59 mg, 0.509 mmol) and heated to 100° C. for 3 h. LCMS showed complete conversion to desired product. The crude mixture was diluted with a small amount of DMF and purified directly by gradient elution on reverse phase HPLC (Waters Sunfire OBD C18, 30×150 mm, 5 μm, MeCN/water with 0.1% TFA modifier, 15-85% gradient over 20 min, 40 mL/min). Pure fractions were concentrated in vacuo to yield 5 mg of 21. Mass calculated for C₁₉H₂₁N₃O₃: 339.2; found: 340.4 (M+H)⁺.

### Example 11

### Preparation of Compound 22

Using procedures similar to those described in Example 10 and appropriate starting materials, the following compound was prepared:

| Com- | | |
|---|---|---|
| pound # | Structure | MS |
| 22 | 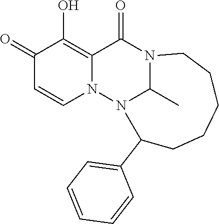 [SMILES: CC1N2CCCCCC(c3ccccc3)N1n1ccc(=O)c(O)c1C2=O] | Mass calculated for C₂₀H₂₃N₃O₃: 353.2; found: 354.4 (M + H)⁺. |

### Example 12

### Preparation of Intermediate Int-12b

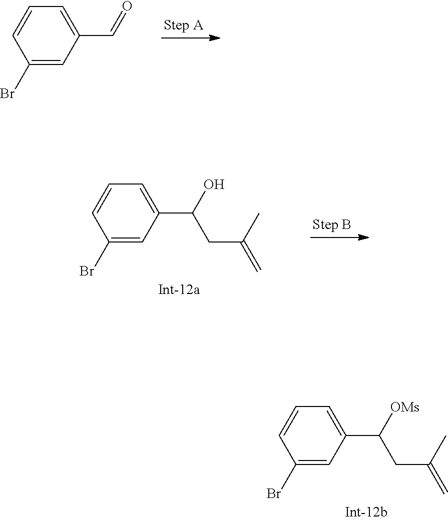 [SMILES: O=Cc1cccc(Br)c1 ; C=C(C)CC(O)c1cccc(Br)c1 ; C=C(C)CC(OS(C)(=O)=O)c1cccc(Br)c1]

### Step A—Synthesis of Compound Int-12a

A solution of 3-bromobenzaldehyde (800 mg, 4.32 mmol) in THF (18.9 ml) was cooled to 0° C. and treated with (2-methylallyl)magnesium chloride (9512 μl, 4.76 mmol) and stirred at 0° C. for 10 min. The mixture was removed from the cold bath and allowed to warm to rt and stir for another 15 min. LCMS showed that the reaction was nearly complete. The mixture was quenched with sat NH₄Cl and extracted with EtOAc twice. The organic phase was dried over Na₂SO₄, filtered and concentrated. The crude material was purified by gradient elution on silica gel (RediSep-Rf-40 g, 0-15% EtOAc/hexanes, 20 minute gradient) to afford Int-12a. Mass calculated for C₁₁H₁₃BrO: 240.0/242.0; found: 223.1/225.1 (M-OH)⁺.

### Step B—Synthesis of Compound Int-12b

A solution of Int-12a (255 mg, 1.058 mmol) in DCM (10.6 mL) was treated with triethylamine (0.360 mL, 2.64 mmol) and methanesulfonyl chloride (0.151 mL, 1.586 mmol) at rt and stirred for 10 min. LCMS shows near complete conversion. The mixture was partitioned between water and DCM. The aqueous phase was back extracted with DCM. The combined organic phases were dried over Na₂SO₄, filtered and concentrated to afford Int-12b. This material was carried forward without further purification.

### Example 13

### Preparation of Compound 23

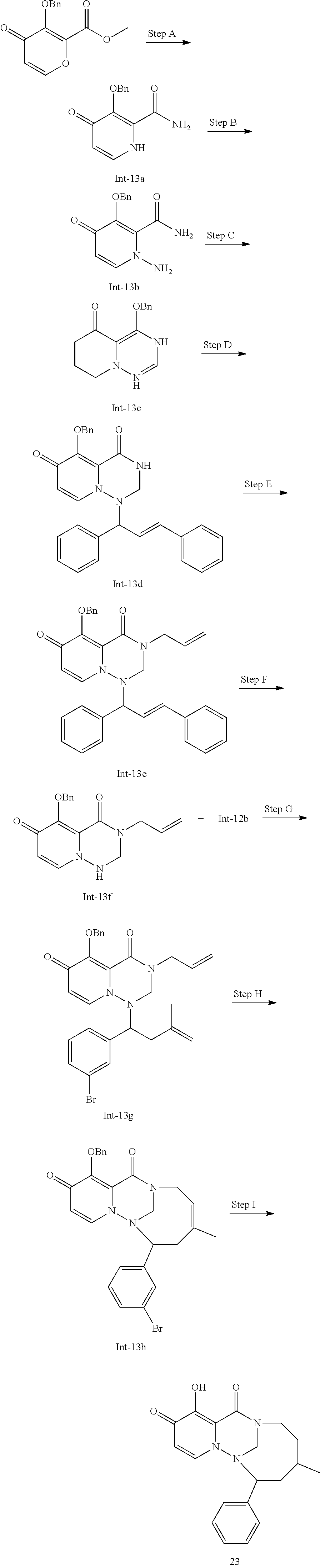

### Step A—Synthesis of Compound Int-13a

A mixture of methyl 3-(benzyloxy)-4-oxo-4H-pyran-2-carboxylate (1.1 g, 4.23 mmol) and ammonium hydroxide (20 ml, 4.23 mmol) in ethanol (20 ml) was stirred at room temperature overnight. LCMS showed that the reaction was complete. The mixture was concentrated in vacuo to afford Int-13a. Mass calculated for C₁₃H₁₂N₂O₃: 244.1; found: 245.0 (M+H)⁺.

### Step B—Synthesis of Compound Int-13b

To a stirred solution of Int-13a (7 g, 28.7 mmol) and Cs₂CO₃ (37.4 g, 115 mmol) in DMF (80 mL) was added O-(2,4-dinitrophenyl)hydroxylamine (11.41 g, 57.3 mmol) at 0° C., then the mixture was stirred at 15° C. for 13 h. LCMS showed the reaction was completed. The mixture was filtered and the filtrate was concentrated under reduced pressure and purified by silica gel column eluting with 10% MeOH/DCM to give Int-13b. Mass calculated for C₁₃H₁₃N₃O₃: 259.1; found: 260.0 (M+H)⁺.

### Step C—Synthesis of Compound Int-13c

A solution of Int-13b (200 mg, 0.771 mmol), formaldehyde (23.16 mg, 0.771 mmol) and acetic acid (4 ml, 0.771 mmol) in DMF (4 mL) was stirred at 100° C. for 2 h. LCMS showed the reaction was complete. The mixture was concentrated under reduced pressure to give Int-13c, which was used for the next step without further purification. Mass calculated for C₁₄H₁₃N₃O₃: 271.1; found: 272.0 (M+H)⁺.

### Step D—Synthesis of Compound Int-13d

A suspension of Int-13c (3 g, 11.06 mmol) and (E)-1,3-diphenylprop-2-en-1-ol (2.79 g, 13.27 mmol) in ethyl acetate (18.4 mL) was treated with 1-propanephosphonic anhydride (50% wt solution in EtOAc) (13.17 ml, 22.12 mmol) and then methanesulfonic acid (1.436 ml, 22.12 mmol) dropwise. The reaction was stirred at rt for 15 min. LCMS showed complete conversion to desired product. The mixture was quenched with sat. aq. NaHCO₃ and the mixture was partitioned between water and EtOAc. The organic phase was dried over Na₂SO₄, filtered and concentrated to afford Int-13d. Mass calculated for C₂₉H₂₅N₃O₃: 463.2; found: 464.4 (M+H)⁺.

### Step E—Synthesis of Compound Int-13e

A solution of Int-13d (5.1 g, 11.00 mmol) in DMF (36.7 mL) was treated with sodium hydride (0.880 g, 22.00 mmol) and allyl bromide (1.915 ml, 22.00 mmol) at rt. The mixture was then heated to 60° C. for 1 h 40 min. LCMS showed near-complete conversion to desired product. The mixture was cooled to rt, quenched with water, and partitioned between DCM and water. The aqueous phase was back-extracted with DCM. The combined organic phases were dried over Na₂SO₄, filtered, and concentrated in vacuo to give Int-13e. Mass calculated for C₃₂H₂₉N₃O₃: 503.2; found: 504.4 (M+H)⁺.

### Step F—Synthesis of Compound Int-13f

The crude residue Int-13e (5.54 g, 11.00 mmol) was dissolved in 4 N HCl in dioxane (20 mL, 80 mmol) and stirred at rt for 1 h. LCMS showed near complete reaction. The reaction was concentrated to dryness and was purified by gradient elution on silica gel (RediSep-Rf-330 g, 0-15% MeOH/DCM, 35 minute gradient) to afford Int-13f. Mass calculated for C₁₇H₁₇N₃O₃: 311.1; found: 312.3 (M+H)⁺.

### Step G—Synthesis of Compound Int-13g

A solution of Int-13f (165 mg, 0.530 mmol) in DMF (2.1 mL) was treated with sodium hydride (76 mg, 3.18 mmol) and stirred for 1 min. Then a solution of freshly made Int-12b (338 mg, 1.060 mmol) in DMF (2.1 mL) was added to the mixture and was stirred at rt for 10 min. LCMS shows minimal conversion to desired product. The mixture was treated two more times with sodium hydride (76 mg, 3.18 mmol) over a 30 min period to push to completion. LCMS showed that the reaction was complete. The mixture was quenched at rt with water and was partitioned between water and EtOAc. The aqueous phase was back-extracted with EtOAc. The combined organic phases were dried over Na₂SO₄, filtered and concentrated. The crude material was purified by gradient elution on silica gel (RediSep-Rf-12 g, 0-10% MeOH/DCM, 10 minute gradient) to afford Int-13g. Mass calculated for C₂₈H₂₈BrN₃O₃: 533.1/535.1; found: 534.3/536.3 (M+H)⁺.

### Step H—Synthesis of Compound Int-13h

A solution of Int-13g (98 mg, 0.183 mmol) in DCE (37 mL) was degassed for 3 min by bubbling N₂ gas through the solution. The solution was treated with Grubbs 2^{nd} generation catalyst (31.1 mg, 0.037 mmol) and stirred at 60° C. for 3 h. LCMS showed conversion to desired product. The mixture was cooled to rt and concentrated in vacuo. The crude material was purified by gradient elution on silica gel (RediSep-Rf-12 g, 0-10% MeOH/DCM, 10 minute gradient) to afford impure material. This impure material was diluted with a small amount of 1:1 MeCN:water and purified directly by gradient elution on reverse phase HPLC (Waters Sunfire OBD C18 30×150 mm, 5 μm, MeCN/water with 0.1% TFA modifier, 5-85% gradient over 20 min, 50 mL/min). Pure fractions were concentrated in vacuo to yield Int-13h. Mass calculated for C₂₆H₂₄BrN₃O₃: 505.1/507.1; found: 506.3/508.3 (M+H)⁺.

### Step I—Synthesis of Compound 23

A solution of Int-13h (25 mg, 0.049 mmol) in 1:1 MeOH:EtOAc (1 mL) was treated with palladium hydroxide (20 wt %) (6.93 mg, 9.87 μmol). The flask was filled with H_{2(g)} and purged vigorously for 10 seconds. The solution was stirred under a H_{2(g)} atmosphere (balloon) at rt for 1 h. LCMS showed that the reaction was complete. The catalyst was filtered off through a syringe filter, washing with MeOH and EtOAc. The filtrate was concentrated and the crude material was diluted with a small amount of 1:1 MeCN:water and purified directly by gradient elution on reverse phase (Waters Sunfire OBD C18 30×150 mm, 5 μm, MeCN/water with 0.1% TFA modifier, 0-60% gradient over 20 min, 40 mL/min). Pure fractions were concentrated in vacuo to yield 23. Mass calculated for C₁₉H₂₁N₃O₃: 339.2; found: 340.3 (M+H)⁺.

### Flu A Neuraminidase Antiviral Assay

A fluorescent assay monitoring the activity of influenza-derived neuraminidase (NA) enzyme in infected cells was developed to evaluate antiviral compounds against Flu A. NA activity enables the release of influenza virions from infected cells but is also functional in cell culture systems that retain infectious virus at the cell surface like Madin-Darby canine kidney (MDCK) epithelial cells which require chemical intervention for release of virus. NA activity can be monitored by the increase in fluorescence of MUNAN (4-methylumbelliferone) released as product from enzymatic substrate cleavage of 2′-(4-methylumbelliferyl)-α-D-N-acetylneuraminic acid (MUNANA). The amount of fluorescence is directly proportional to the amount of NA enzyme activity which increases with viral replication.

MDCK (Sigma) cells are incubated at 37° C. in an atmosphere of 5% CO₂, and >85% humidity in growth medium of DMEM with Glutamax and pyruvate (Thermo Fisher) with 5% heat-inactivated fetal bovine serum (Thermo Fisher), and 1% Pen-Strep (Thermo-Fisher). On the day of assay, cells are washed with 15-20 ml PBS (Thermo Fisher) followed by the addition of 1.5 ml 0.25% Trypsin-EDTA (Thermo Fisher) solution and incubation at 37° C. for 2-5 min. After the cells have dislodged from the plate, 6-8 mL of growth medium is added to resuspend cells. Cells are counted in a ViCell Counter (Beckman), and the cell density is adjusted to 80,000 cells/mL by adding growth medium. FLU A (PR/8/34) at 3.4E+08 pfu/ml is diluted 1:25,000 in to the MDCK cell suspension.

Compounds diluted in DMSO are titrated (10-point, 3-fold dilution) and added by acoustic dispenser (200 nL, Labcyte Echo) into a 384-well black polystyrene tissue-culture treated microplate (Corning). The cell and virus suspension (25 μL) is dispensed into each well of the assay plate. Plates are briefly centrifuged to (300 rpm×30 s) and incubated at 37° C. in an atmosphere of 5% CO₂, and >85% humidity for 48 h. For detection of NA activity, the MUNANA substrate (MP Biomedical) is diluted in dH₂O at a concentration of 2.5 mM. The substrate is further diluted in assay buffer (66.6 mM MES, 8 mM CaCl₂), pH 6.5) to a concentration of 200 μM. A volume of 6 μl of the substrate dilution is added to each well of the assay plate, shaken for 1 min to mix and returned to the incubator for 1 h at 37° C. Then, 25 μL of MUNANA Stop Solution (0.2 M sodium carbonate, Fisher) in dH₂O was added to each well followed by shaking for 1 min. An Envision plate reader (Perkin Elmer) was used to measure the fluorescence intensity (Ex=355 nm, Em=460 nm) of each well.

Raw data from each test well is normalized to the average signal of 100% inhibited wells (Max Effect; 100% inhibition) and virus infected cells only (Min Effect; 0% inhibition) to calculate % inhibition using the following formula: % inhibition=100*(Test Cmpd−Max Effect)/(Min Effect−Max Effect). Data are analyzed using ActivityBase (IDBS), and dose-response curves were generated by plotting percent inhibition (Y-axis) vs. Log₁₀ compound concentrations (X-axis). IC₅₀ values are calculated using a non-linear regression, four-parameters sigmoidal dose-response model.

The compounds of the instant invention were tested in the assay described and the results appear in the table below.

| Examples | Flu A EC₅₀ (nM) |
|---|---|
| 1 | 143 |
| 2 | 72 |
| 3 | 133 |
| 4 | 1690 |
| 5 | 92 |
| 6 | 2620 |
| 7 | 4880 |
| 8 | 3900 |
| 9 | 578 |
| 10 | 577 |
| 11 | 966 |
| 12 | 6290 |
| 13 | 310 |
| 14 | 927 |
| 15 | 460 |
| 16 | 164 |
| 17 | 193 |
| 18 | 1460 |
| 19 | 229 |
| 20 | 171 |
| 21 | 98 |
| 22 | 228 |
| 23 | 4410 |

1. A compound of the formula 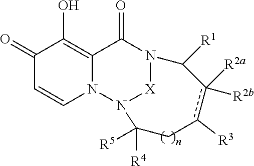
wherein:
-- is an optional double bond;
X is CHR⁶;
R¹ is selected from the group consisting of hydrogen, halo, hydroxy, C₁₋₆ alkyl and C₃₋₆ cycloalkyl;
R^{2a} is selected from the group consisting of hydrogen, halo, hydroxy and C₁₋₆ alkyl;
R^{2b} is hydrogen or halo, but when --- is a double bond, R^{2b} is absent;
R³ is selected from the group consisting of hydrogen, halo, hydroxy and C₁₋₆ alkyl;
R⁴ is selected from the group consisting of hydrogen and C₁₋₆ alkyl;
R⁵ is aryl, which may be monocyclic or bicyclic, which is optionally substituted with one to three substituents independently selected from the group consisting of halo, R^{x} and OR^{x};
R⁶ is hydrogen or C₁₋₃ alkyl;
R^{x} is selected from the group consisting of hydrogen and C₁₋₆ alkyl, wherein said alkyl is optionally substituted with one to three halo; and
n is one or two;
or a pharmaceutically acceptable salt thereof.

2. The compound of claim 1, wherein R¹ is hydrogen, fluoro, methyl or cyclopropyl; or a pharmaceutically acceptable salt thereof.

3. The compound of claim 1, wherein R^{2a} is hydrogen or fluoro; or a pharmaceutically acceptable salt thereof.

4. The compound of claim 1, wherein R³ is hydrogen, methyl or hydroxy; or a pharmaceutically acceptable salt thereof.

5. The compound of claim 1, wherein R⁴ is hydrogen or methyl; or a pharmaceutically acceptable salt thereof.

6. The compound of claim 1, wherein R⁵ is phenyl or naphthalenyl, wherein said phenyl and naphthalenyl are optionally substituted with halo; or a pharmaceutically acceptable salt thereof.

7. The compound of claim 1, wherein R⁶ is hydrogen or methyl; or a pharmaceutically acceptable salt thereof.

8. A compound selected from compounds 1-23, or a pharmaceutically acceptable salt thereof.

9. A pharmaceutical composition comprising a compound of claim 1 or a pharmaceutically acceptable salt thereof and a pharmaceutically acceptable carrier.

10. A method for treating a disease caused by a virus having a cap-dependent endonuclease comprising administering a composition of claim 9 to a mammal in need of thereof.

11. A method for inhibiting cap-dependent endonuclease in a virus comprising administering a composition of claim 9, to a mammal in need thereof.

12. A method for treating influenza in a mammal comprising administering a composition of claim 9, to a mammal in need thereof.

13. A method of preventing influenza in a mammal comprising administering a composition of claim 9, to a mammal in need thereof.

14. The method of claim 12, wherein the mammal is human.

15. The method of claim 13, wherein the mammal is human.

16.-17. (canceled)
